(19) United States
(12) Patent Application Publication
(10) Pub. No.: US 2006/0051380 A1
(43) Pub. Date: Mar. 9, 2006

(54) Methods and compositions for the targeting of a systemic immune response to specific organs or tissues

(75) Inventors: Richard D Schulick, Baltimore, MD (US); Drew M. Pardoll, Brookeville, MD (US); Ajay Jain, Baltimore, MD (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Johns Hopkins University, Baltimore, MD (US)

(21) Appl. No.: 10/504,039

(22) PCT Filed: Feb. 6, 2003
(86) PCT No.: PCT/US03/03650

§ 371 Date: Aug. 18, 2005

Related U.S. Application Data
(60) Provisional application No. 60/354,564, filed on Feb. 6, 2002.

(51) Int. Cl. A61K 39/00; A61K 48/00

(52) U.S. Cl. 424/277.1; 424/93.2

(57) ABSTRACT

The invention provides methods and compositions for targeting a separately generated immune response to a specific organ or tissue, e.g. one affected by cancer, using one or more agents with a tropism for the organ or tissue or that can be specifically localized to the desired organ or tissue. For example, the invention provides methods ands compositions for treating liver metastases from colorectal cancer using a combination of a granulocyte/macrophage colony stimulating factor (GM-CSF) augmented tumor cell vaccination and Listeria monocytogenes (LM) infection.

### RELATED APPLICATIONS

This is a 371 of PCT/US03/03650, filed on Feb. 6, 2003, which claims the benefit of priority to U.S. Provisional Patent Application No. 60/354,564, filed on Feb. 6, 2002, which applications are hereby incorporated by reference in their entirety.

### GOVERNMENT SUPPORT

This work was supported by a Public Health Service (NIH-NCI GI SPORE Grant No. 5P50 CA 62924-08) as well as a Clinician Scientist Award from Johns Hopkins University. The government may have certain rights in the invention.

### 1. BACKGROUND OF THE INVENTION

Cancer continues to be one of the most devastating health problems in the world today, affecting some one in five individuals in the United States. Research has led to the discovery of many different types of therapies, including cytotoxic agents commonly employed in chemotherapy such as anti-metabolic agents which interfere with microtubule formation, alkylating agents, platinum-based agents, anthracyclines, antibiotic agents, topoisomerase inhibitors, and others. In addition, the more traditional surgical and radiation therapies have been refined, while cutting edge treatments involving immune modulation and gene therapy have been developed. Nevertheless, although thousands of potential anticancer agents have been evaluated, the treatment of human cancer remains fraught with complications and side effects which often present an array of suboptimal treatment choices.

One interesting novel non-chemical approach to treating cancer is based upon the observation that rapidly developing tumors possess irregular vascular organization resulting in temporally and spatially heterogenous blood flow and resulting hypoxic regions. This heterogeneity of tumor blood flow hinders the delivery of blood-borne chemotherapeutics to cancer cells and these conditions reduce the effectiveness of radiation and chemotherapeutic agents and tends to select for cancer cells that are more aggressive and metastatic (see Brown and Giaccia (1998) Cancer Res 58: 1408-1416). Ironically, these same hypoxic conditions lead to the advantageous localization of non-pathogenic, anaerobic bacteria that localize and can cause lysis from within transplanted tumors (see e.g. Carey et al. (1967) Eur. J. Cancer 3: 37-46 describing the use of Clostridium to treat tumors). Such early efforts were not very successful. More recently Dang et al. ((2001) PNAS USA 98: 15155-160; and commentary by Jain and Forbes (2001) PNAS USA 98: 16748-750) screened some 26 strains of bacteria for their ability to uniformly infect and spread through the poorly vascularized regions of the tumor. One strain of Clostridium novyi was particularly effective and was genetically modified to eliminate its encoded lethal toxin. The resulting infected tumors suffered bacterial-induced necrosis, but did not completely eliminate peripheral viable and vascularized tumor cells—which had to be treated with conventional chemotherapeutics (the combination chemotherapeutic/bacteriolytic therapy being termed “COBALT”). In related approaches Lemmon et al. ((1997) Gene Ther 4: 791-96) have described the engineering of anaerobic bacteria as a gene delivery system that is controlled by the tumor microenvironment, and Theys et al. ((2001) Cancer Gene Ther 8: 294-97) have described the specific targeting of cytosine deaminase to solid tumors by engineered Clostricium acetobutylicum.

Despite the development of these novel bacteriolytic therapies and the great number of anti-neoplastic agents that are available for use in the clinical treatment of cancer, a need still exists for more effective regimens for treating cancer. Accordingly, the need exists for an improved cancer treatments, especially malignant solid tumors affecting specific organs or tissues and their resulting metastatic tumors.

A developing alternative approach to chemotherapeutic treatments for cancer and neoplasms has been the use of tumor vaccines which are based on weakly immunogenic specific tumor antigens admixed with adjuvants in order to elicit, restore or augment antitumor immune responses against residual or metastatic tumor cells. Tumor vaccines-mediated therapy involves the activation of cellular toxicity in the targeted tumor cells (see Nawrocki and Mackiewicz (1999) Cancer Treat Rev 25: 29-46 for review). Several HLA-restricted specific tumor antigens recognized by cytotoxic T-cells have been characterized. The first generation of tumor vaccines include those made of whole cancer cells or tumor cell lysates together with non-specific adjuvants. Novel second generation tumor vaccines employ genetically modified tumor cells, antigen presenting cells (dendritic cells) or recombinant tumor antigens (e.g. DNA tumor vaccines as further defined below). Tumor cells may be modified to enhance their efficacy in eliciting anti-tumor immune responses by genetic modification with genes encoding molecules that provide signals for cytotoxic T-cells required for recognition and killing of cancer cells such as B7 costimulatory molecule, HLA proteins and genes of different cytokines (e.g. granulocyte-macrophage colony stimulating factor or GM-CSF).

Another tumor vaccine strategy is based on the observation that inoculation of a plasmid containing cDNA encoding a tumor protein antigen leads to strong and long-lived humoral and cell-mediated immune responses to the tumor antigen. Accordingly, if an effective tumor antigen can be identified it is possible to insert the DNA sequence coding for the tumor protein antigen into a carrier genome (e.g. a plasmid or alphavirus) to elicit an anti-tumor immune response. Such DNA vaccines based upon specific tumor antigens are known as DNA tumor vaccines (or DNA cancer vaccines). It is speculated that certain bone marrow-derived profession antigen presenting cells (APCs) are transfected by the plasmid and the cDNA is transcribed and translated into immunogenic protein that elicits specific responses. In a related strategy, so called naked DNA is injected directly into the host to produce an immune response. Naked DNA includes simple bacterial plasmids which are injected directly into the host. The ability of DNA vaccines to deliver precise and specific nucleotide sequences representing target genes such as the ALVAC gp100 gene for melanoma and the ALVAC CEA-B7.1 gene for colorectal cancer and specific protein fragments such as the HER2/Neu peptide found in breast cancer cells have been studied as a potential mean with which to induce an immune response (see e.g. Tartaglia et al. (2001) Vaccine 19: 2571-5; Knutson et al. (2001) J Clin Invest 107: 477-84; Chen et al. (2001) Gene Ther 8: 316-23; and Sivanandham et al. (1998) Cancer Immunol Immunother 46: 261-7). Unfortunately, intramuscular injection of DNA frequently fails to generate a vigorous immune response, although transdermal or intradermal delivery of DNA may be more effective. For example, a clinical trial of transdermally delivered microscopic gold beads coated with hepatitis B antigen-encoding DNA generated protective levels of antibodies to the antigen (see Poland et al. The Fourth Annual Conference on Vaccine Research, Arlington, Va. (Apr. 23-25, 2001 (www.nfid.org/conferences/vaccine01/abstracts/abss37-40.pdf)) DNA vaccines, where effective, provide a unique approach for eliciting strong cytotoxic T lymphocyte (CTL) because the DNA-encoded proteins are synthesized in the cytoxol of transfected cells. Furthermore, bacterial plasmids are rich in unmethylated CpG nucleotides and are recognized as foreign by macrophages and elicit an innate immune response that enhances adaptive immunity. Accordingly, plasmid DNA vaccines are effective even when administered without adjuvants. Furthermore, the cDNAs expressed by such vaccines are readily manipulated to express many diverse antigens and provide for the ability to coexpress other proteins that may enhance the immune response (e.g. cytokines and costimulators). Nevertheless, specific DNA vaccines must developed through testing and proof of efficacy. This is particularly true in the case of DNA vaccines applications for the treatment of cancer, because even highly-expressed tumor specific antigens are not always effective targets for DNA cancer vaccine immunotherapy. Accordingly, as only the first step in DNA tumor vaccine development, effective, generally surface-expressed tumor-specific antigens must be identified.

Such novel tumor vaccine strategies have produced specific anti-tumor immune responses and objective clinical responses. Nevertheless, such tumor vaccine strategies are neither completely nor consistently successful and, accordingly, improved methods for the immunological treatment of cancer are needed.

### 2. SUMMARY OF THE INVENTION

In general, the invention provides a method of generating a systemic immune response against an organ or tissue-specific disease or condition in a subject by administering a therapeutically effective amount of a vaccine which generates an immune response against the organ or tissue-specific disease or condition in conjunction with the administration of an agent that tropically localizes or is administered directly to the organ or tissue and that generates a localized immune response at the organ or tissue. In preferred embodiments, the organ or tissue-specific disease or condition is a tumor or cancerous growth and the vaccine is a tumor vaccine. In another preferred embodiment, the vaccine is an attenuated tumor cell line expressing GM-CSF. In another embodiment, the agent that tropically localizes to the organ or tissue is a virus, a bacterium, a yeast or a fungus with a natural tropism for the specific organ or tissue. Preferably, the agent that tropically localizes is an attenuated strain of Listeria monocytogenes. Still more preferably, the organism tropically localizes to neovascular endothelium.

In certain embodiments, the agent is genetically engineered to tropically localize to the organ or tissue and is an engineered virus, bacterium, yeast or fungus. Preferably, the genetically engineered organism expresses a ligand for a receptor expressed by the organ or tissue. In another preferred embodiment, the ligand for the organ or tissue receptor has been fused to an envelope or coat protein of the organism. Still more preferably, the organ or tissue targeted is neovascular endothelium. Accordingly, in preferred embodiments, the genetically engineered organism expresses a ligand for a receptor expressed by neovascular endothelium.

In other embodiments, the agent is an organism without natural tropism that is administered directly to the organ or tissue by a physical means selected such as by direct injection, percutaneous catheter, surgery, and closed loop perfusion. In preferred embodiments, the organ or tissue administered to is the lungs and the method of administration is inhalation. In another preferred embodiment, the organ or tissue targeted is the gastrointestinal tract and the method of administration is ingestion.

In another embodiment, the agent that tropically localizes or is administered directly to the organ or tissue is a genetically engineered organism that produces an activator of immunity or inflammation such as a chemokine, a cytokine, or an adhesion molecule. Preferably, the agent that tropically localizes or is administered directly to the organ or tissue is an inflammatory agent.

In another preferred embodiment, the invention provides a method of treating a tumor or cancerous growth localized to a tissue or organ in a subject by administering a therapeutically effective amount of a tumor vaccine which generates an immune response against the tumor or cancerous growth in conjunction with an agent that tropically localizes or is administered directly to the organ or tissue and that generates a localized immune response at the organ or tissue. In preferred embodiments of this aspect of the invention, the tumor or cancerous growth is a hepatic tumor, the tumor vaccine is a GM-CSF secreting whole tumor cell vaccine, and the agent that tropically localizes to the affected organ or tissue is an attenuated strain of Listeria monocytogenes. In other preferred embodiments, the tumor vaccine is a DNA tumor vaccine.

The invention further provides formulations for generating a systemic immune response against an organ or tissue-specific disease or condition in a subject comprising a therapeutically effective amount of a vaccine which generates an immune response against the organ or tissue-specific disease or condition; and an agent that tropically localizes or is administered directly to the organ or tissue and that generates a localized immune response at the organ or tissue. Preferably, the formulation is for treating a tumor or cancerous growth localized to a tissue or organ in a subject comprising a therapeutically effective amount of a tumor vaccine which generates an immune response against the tumor or cancerous growth; and an agent that tropically localizes or is administered directly to the organ or tissue and that generates a localized immune response at the organ or tissue. Still more preferably, the agent that tropically localizes or is administered directly to the organ or tissue and that generates a localized immune response at the organ or tissue is an attenuated bacteria. Most preferably, the attenuated bacteria is an HIV-gag attenuated Listeria monocytogenes.

The invention further provides for kits for generating a systemic immune response against an organ or tissue-specific disease or condition in a subject comprising: a vaccine which generates an immune response against the organ or tissue-specific disease or condition; and an agent that tropically localizes or is administered directly to the organ or tissue and that generates a localized immune response at the organ or tissue. Preferably, the kit is for treating a tumor or cancerous growth localized to a tissue or organ in a subject and includes a tumor vaccine which generates an immune response against the tumor or cancerous growth; and an agent that tropically localizes or is administered directly to the organ or tissue and that generates a localized immune response at the organ or tissue.

### 3. BRIEF DESCRIPTION OF THE FIGURES

FIG. 1 shows the design of the murine hepatic metastasis model procedure in which the spleen is divided into two hemi-spleens.

FIG. 2 shows injection of CT26 murine colorectal cancer tumor cells into one of the hemi-spleens to form tumor deposits in the liver and the injected hemi-spleen surgically removed to leave a functional hemi-spleen free of tumor cells.

FIG. 3 shows the histology of the murine CT26 hepatic metastasis model.

FIG. 4 shows control and experimental gross liver specimens four weeks following challenge in the murine CT26 hepatic metastasis model.

FIG. 5 shows the temporal effect of vaccination with the irradiated GM-CSF-expressing tumor whole cell vaccine on mouse survival in the CT26 hepatic metastasis model.

FIG. 6 shows improved survival of mice from hepatic metastasis with the combination treatment of GM-CSF tumor vaccine and attenuated Listeria monocytogenes infection.

FIG. 7 shows that Listeria monocytogenes tumor vaccine augmentation is specific to the liver and not the lung.

FIG. 8 shows a comparison of liver infiltrating CD8 T-cell specificity for AH1 tumor antigen.

FIG. 9 shows a comparison of survival of hepatic tumor bearing mice treated with either vaccine, Listeria or a combination of vaccine and Listeria.

FIG. 10 shows that Listeria monocytogenes tumor vaccine augmentation is specific to the liver and not pulmonary tumors.

FIG. 11 shows that double CD8 panning increases the purity of CD8 lymphocytes isolated from mouse livers.

FIG. 12 (A-C) shows the results of analysis of liver infiltrating, tumor-specific CD8 T-cell numbers from mice in the different treatment groups.

FIG. 13 (A-C) shows the results of a second analysis of liver infiltrating, tumor-specific CD8 T-cell numbers from mice in the different treatment groups.

FIG. 14 (A-E) shows the results of a third analysis of liver infiltrating, tumor-specific CD8 T-cell numbers from mice in the different treatment groups.

FIG. 15 shows RT-PCR analysis of liver infiltrating, AH1-specific CD8 T-cells for IFN-gamma and IL-10.

### 4. DETAILED DESCRIPTION OF THE INVENTION

4.1. General

In general, the invention provides methods and compositions for targeting a separately generated immune response to a specific organ or tissue, e.g. one affected by cancer, using one or more agents with a tropism for the organ or tissue or that can be specifically localized to the desired organ or tissue. The invention is particularly beneficial where methods and compositions for generating the specifically-targeted immune response are used in combination with a second immunologic agent, e.g. a vaccine, that generates a generalized immunological response. In preferred embodiments, the invention provides means for avoiding potential problems associated with a systemically generated, generalized immunologic response, such as occur with vaccines. In particular, such immunological responses are unfocused and do not target a specific organ or tissue. In certain other instances, the unfocused immunological response cannot gain access to the desired specific target organ or tissue. In still other instances, the unfocused immunological response is not strong enough to cause a desired affect in a specific organ or tissue even if it can gain access or can be focused. The invention provides agents and methods of their that facilitate tissue and/or organ-specific tropism to the immune response—e.g. the immune response generated by a vaccine (such as a tumor vaccine).

In preferred embodiments, the invention provides agents that possess a tropism for a specific organ(s) or tissue(s) that: focus the biologic response to a specific organ or tissue by mechanisms that help the appropriate cells track to the correct location; change the local microenvironment to allow the biologic response access to that location; and that nurture or amplify the biologic response once it has locally reached the target.

In broadly preferred embodiments the invention provides the combination of any approach to generate a systemic immune response with means for providing a tissue or organ-specific immune response. Such means for generating a focused, tissue or organ-tropic immune response for use in the invention include: any infectious agent such as a virus, bacterium, yeast, or fungus with a natural tropism for a specific organ or tissue; any infections agent in which the tropism for a specific organ or tissue has been engineered (for example by splicing the ligand for an organ or tissue specific receptor into an envelope or coat protein of the organism); any organism with natural or engineered tropism for a neovascular endothelium; placement of an organism in a particular organ or tissue by physical means such as direct injection, percutaneous catheter, surgery, or closed loop perfusion to target an organism without natural tropism; inhalation or ingestion of an organism to target the lungs or gastrointestinal tract; or, in another preferred embodiment the use of all the above methods in which the organism is genetically engineered to produce chemokines, cytokines, adhesion molecules, or other activators of immunity or inflammation. In another preferred embodiment, the tropic agent is a nonliving inflammatory agent (e.g. a small molecules) that naturally or via engineering generates a tropic immune response.

In broad terms, the invention targets a separately generated immune response to a specific organ or tissue with the use of an agent that has a tropism for that organ or tissue or that can be specifically placed at that site. In a particularly preferred embodiment, a liver metastases from a colorectal cancer is treated with a combination of a granulocyte/macrophage colony stimulating factor (GM-CSF) augmented tumor cell vaccination and Listeria monocytogene (LM) infection. While not wishing to be limited to a particular mechanism of action, the invention provides for vaccination with a GM-CSF or equivalently augmented tumor cell which causes a systemic T cell mediated immune response within the subject and infection with an attenuated LM, which preferentially infects the liver, focuses the systemic vaccine induced immune response on the liver by changing the local environment, chemokine release, cytokine release, adhesion molecule expression, or vascular permeability within the liver. The net result is enhanced anti-tumor immunity against the liver metastases. The invention applies broadly to the combination of any approach that generates a systemic immune response and an organ or tissue specific inflammatory stimulus generated by an organism with selective homing properties or regional installation by physical means.

4.2. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “aberrant activity”, as applied to an activity of a polypeptide refers to an activity which differs from the activity, of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example, an aberrant polypeptide can interact with a different target peptide. A cell can have an aberrant polypeptide activity due to overexpression or underexpression of the gene encoding the polypeptide.

The term “agonist”, as used herein, is meant to refer to an agent that mimics or upregulates (e.g. potentiates or supplements) a bioactivity. A polypeptide agonist can be a wild-type protein or derivative thereof having at least one bioactivity of the wild-type polypeptide. A polypeptide therapeutic can also be a compound that upregulates expression of a polypeptide-encoding gene or which increases at least one bioactivity of a polypeptide. An agonist can also be a compound which increases the interaction of a polypeptide with another molecule, thereby promoting.

The term “allele”, which is used interchangeably herein with “allelic variant”, refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. Frequently occurring sequence variations include transition mutations (i.e. purine to purine substitutions and pyrimidine to pyrimidine substitutions, e.g. A to G or C to T), transversion mutations (i.e. purine to pyrimidine and pyrimidine to purine substitutions, e.g. A to T or C to G), and alteration in repetitive DNA sequences (e.g. expansions and contractions of trinucleotide repeat and other tandem repeat sequences). An allele of a gene can also be a form of a gene containing a mutation. The term “allelic variant of a polymorphic region of a gene” refers to a region of a gene having one or several nucleotide sequence differences found in that region of the gene in certain individuals.

“Antagonist” as used herein is meant to refer to an agent that downregulates (e.g. suppresses or inhibits) at least one bioactivity. An antagonist can be a compound which inhibits or decreases the interaction between a protein and another molecule, e.g., a ligand and a receptor. An antagonist can also be a compound that down-regulates expression of a gene or which reduces the amount of gene product protein present. The ligand antagonist can be a dominant negative form of a ligand polypeptide, e.g., a form of a ligand polypeptide which is capable of interacting with a target peptide. An antagonist can also be a compound that interferes with a protein-dependent signal transduction pathway.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

The term “anti-tumor activity” or “antineoplastic activity” refers to the ability of a substance or composition to block the proliferation of, or to induce the death of tumor cells which interact with that substance or composition.

A disease, disorder, or condition “associated with” or “characterized by” an aberrant expression of a nucleic acid refers to a disease, disorder, or condition in a subject which is caused by, contributed to by, or causative of an aberrant level of expression of a nucleic acid.

As used herein the term “bioactive fragment of a polypeptide” refers to a fragment of a full-length polypeptide, wherein the fragment specifically mimics or antagonizes the activity of a wild-type polypeptide. The bioactive fragment preferably is a fragment capable of interacting with the specific polypeptide's receptor(s).

“Biological activity” or “bioactivity” or “activity” or “biological function”, which are used interchangeably, for the purposes herein means an effector or antigenic function that is directly or indirectly performed by a polypeptide (whether in its native or denatured conformation), or by any subsequence thereof. Biological activities include binding to a target peptide. A target polypeptide bioactivity can be modulated by directly affecting the target polypeptide. Alternatively, a target polypeptide bioactivity can be modulated by modulating the level of the target polypeptide, such as by modulating expression of the target polypeptide-encoding gene.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject polypeptides with a second amino acid sequence defining a domain (e.g. polypeptide portion) foreign to and not substantially homologous with any domain of the polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-polypeptide-Y, wherein “polypeptide” represents a portion or all of a protein of interest and X and Y are independently absent or represent amino acid sequences which are not related to the protein sequence in an organism, including naturally occurring mutants.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “dendritic cell” refers to any of various accessory cells that serve as antigen-presenting cells (APCs) in the induction of an immune response. As used herein, the term “dendritic cell” includes both interdigitating dendritic cells which are present in the interstitium of most organs and are abundant in T cell-rich areas of the lymph nodes and spleen, as well as throughout the epidermis of the skin, where they are also referred to as Langerhans cells. The interdigitating dendritic cells arise from marrow precursor cells and are related in lineage to mononuclear phagocytes.

As is well known, genes may exist in single or multiple copies within the genome of an individual. Such duplicate genes may be identical or may have certain modifications, including nucleotide substitutions, additions or deletions, which all still code for polypeptides having substantially the same activity. For example, the term “DNA sequence encoding an antigen polypeptide” may thus refer to one or more antigen genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The term “epitope” (or antigenic determinant) is defined as the part of a molecule that combines with a single antigen binding site on an antibody molecule. A single epitope is recognized by a monoclonal antibody (mAb), while multiple epitopes are normally recognized by polyclonal antibodies (Ab).

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids of the invention due to the degeneracy of the genetic code.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the subject gene in genomic DNA, more preferably no more than 1.5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g., based on deletion of at least a portion of the gene, replacement of at least a portion of the gene with a second sequence, introduction of stop codons, the mutation of bases encoding critical amino acids, or the removal of an intron junction, etc.). In preferred embodiments, the “knock-out” gene locus corresponding to the modified endogenous gene no longer encodes a functional polypeptide activity and is said to be a “null” allele. Accordingly, knock-out transgenic animals of the present invention include those carrying one null gene mutation, as well as those carrying two null gene mutations.

A “knock-out construct” refers to a nucleic acid sequence that can be used to decrease or suppress expression of a protein encoded by endogenous DNA sequences in a cell. In a simple example, the knock-out construct is comprised of a gene with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: gene 5′/neo/gene 3′, where gene 5′ and gene 3′, refer to genomic or cDNA sequences which are, respectively, upstream and downstream relative to a portion of the gene and where neo refers to a neomycin resistance gene. In another knock-out construct, a second selectable marker is added in a flanking position so that the gene can be represented as: gene/neo/gene/TK, where TK is a thymidine kinase gene which can be added to either the gene 5′ or the gene 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant genes of the invention is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a sequence shown in one of the DNA sequences of the invention. Nucleic acids at least 90%, more preferably 95%, and most preferably at least about 98-99% identical with a nucleic sequence represented in one of the DNA sequences of the invention are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

The term “polypeptide binding partner” or “polypeptide BP” refers to various cell proteins which bind to a specified polypeptide of the invention.

The term “recombinant protein” refers to a polypeptide of the present invention which is produced by recombinant DNA techniques, wherein generally, DNA encoding a particular polypeptide is inserted into a suitable expression vector which is in turn used to transform a host cell to produce the heterologous protein. Moreover, the phrase “derived from”, with respect to a particular recombinant gene, is meant to include within the meaning of “recombinant protein” those proteins having an amino acid sequence of a particular native polypeptide, or an amino acid sequence similar thereto which is generated by mutations including substitutions and deletions (including truncation) of a naturally occurring form of the polypeptide.

The phrases “parenteral administration” and “administered parenterally” as used herein means modes of administration other than enteral and tropical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The term “stem cell” means a pluripotent cell capable of differentiating into cells of the multiple types of lineages.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate gene, preferably a mammalian gene.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The term “transfected stem cell” is meant a stem cell into which exogenous DNA or an exogenous DNA gene has been introduced by retroviral infection or other means well known to those of ordinary skill in the art.

The term “ex vivo gene therapy” is meant the in vitro transfection or retroviral infection of stem cells to form transfected stem cells prior to introducing the transfected stem cells into a mammal.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the FasL genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of a polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide of the invention (e.g. a gene encoding an antigen or an APC immunostimulatory activity) or, in the case of anti-sense expression from the transferred gene, the expression of a naturally-occurring form of the particular target polypeptide is disrupted.

As used herein, the term “transgene” means a nucleic acid sequence (encoding, e.g., one of the tumor antigen or APC immunostimulatory polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a polypeptide for use in the invention, e.g. either agonistic or antagonistic forms. However, transgenic animals in which a recombinant target gene is silent are also contemplated, as for example, the FLP or CRE recombinase dependent constructs described below. Moreover, “transgenic animal” also includes those recombinant animals in which gene disruption of one or more target genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.3. Vaccines

The invention provides for vaccines, particularly cancer (i.e. tumor) vaccines for use in for generating a generalized systemic immune response to a tissue or organ (e.g. one affected by neoplastic transformation and expressing a tumor cell antigen(s) which can be targeted by the vaccine). Methods and compositions for vaccine technology are known in the art and described, e.g. in U.S. Pat. Nos. 6,511,667; 6,503,503; 6,500,435; 6,488,934; 6,488,926; 6,479,056; 6,472,375; 6,455,492, 6,432,925; and 6,416,764, the contents of each which is incorporated herein by reference. Where the invention provides for tumor or cancer vaccines for generating a generalized anti-tumor systemic immune response, methods and compositions for tumor or cancer vaccine technology are know in the art and as described in, e.g., U.S. Pat. Nos. 6,458,585; 6,432,925; 6,344,198; 6,338,853; 6,377,195; 6,316,256; 6,168,946; 6,106,829; 6,080,722; 5,993,829; 5,861,494; 5,786,204; 5,750,102; 5,733,748; 5,705,151; 5,478,556; 5,290,551 and 5,030,621, the contents of each of which is incorporated herein by in their entirety.

In general, cancer or tumor vaccines may augment already established tumor immunity, are far more specific against the tumor than cytokine therapy and have little or no toxicity, and thus may easily be combined with other types of immunotherapy (see . They also elicit immunological memory, which may check recurrence of the tumor. Melanoma vaccines have received the most attention thus far. Among the several cancer vaccines used are whole cell lysates, such as Melacine, hapten-treated autologous melanoma cells (M-Vax) and irradiated allogeneic cells (CancerVax). Regressions of metastatic nodules have been noted with each preparation. Controlled trials of Melacine indicate prolongation of survival in patients with resected stage IIB disease, particularly those with one or more of the following HLA class I alleles: HLA-A2 or -A28 (-A6802), HLA-B12, -44 or -45, and HLA-C3. A combination of interferon-alpha2b and Melacine appears to enhance the anti-tumor response in advanced (stage IV) disease, and is being tested in a large randomized controlled trial in resected stage III disease. An irradiated autologous colon carcinoma vaccine has improved relapse-free survival in resected stage II disease (Dukes B) in a controlled trial. Second-generation whole cell vaccines include those incorporating genes such as GM-CSF or CD80 (B7-1) to improve immunogenicity, and the use of immunogenic cell membranes such as large multivalent immunogen (LMI). Upregulation of HLA class II molecules and concomitant inhibition of the Ii molecule are also being explored as a strategy for improved presentation of tumor-associated antigens in vaccines. Complex whole cell-derived vaccines have given clinically superior responses compared to vaccines containing well-defined antigens, such as peptides or gangliosides; however, well-defined vaccines may be theoretically more desirable because of their reproducibility.

The goal of cancer treatment is to develop modalities that specifically target tumor cells, thereby avoiding unnecessary side effects to normal tissue. Vaccine strategies that result in the activation of the immune system specifically against proteins expressed by a cancer have the potential to be effective treatment for this purpose. An early vaccine approach that was developed by our group involves the insertion of the granulocyte-macrophage colony stimulating factor (GM-CSF) gene (see e.g. GenBank Accession No. NM_{—}000758 and U.S. Pat. No. 5,641,663, the contents of which are incorporated herein in their entirety) into cancer cells that are then used to immunize patients. These genetically modified tumor cells produce the immune activating protein GM-CSF in the local environment of the tumor cells, specifically activating the patient's T cells to eradicate cancer at metastatic sites. Many studies have demonstrated that this vaccine can cure mice of cancer. This approach can also activate an immune response in patients with renal cell carcinoma and possibly in pancreatic cancer (see e.g. Jaffee et al. (1999) Ann NY Acad Sci 886: 67-72).

Methods and compositions for whole cell tumor or cancer vaccines are known in the art (see e.g. Ward et al. (2002) Cancer Immunol Immunother 2002 September; 51(7):351-7 for review). Briefly, whole tumor cells may generate efficient immunity despite the fact that the immune system is tolerant of certain tumor antigens as they may be expressed by normal tissues, or presented in a non-stimulatory context without co-stimulation. Tumors may also produce immunosuppressive molecules such as IL-10, transforming growth factor- and CD95L. The breaking of tolerance and the overcoming of immune suppression may need a potent and specific immune stimulus. Whole tumor cells are able to provide the antigen source, but additional stimuli such as those provided by immunological adjuvants may be necessary to overcome the induction of tumor-specific T cell anergy.

The process by which tumor cells die, or are made to die by therapy, has also been highlighted as being important in the generation of immune responses against tumor antigens. Apoptotic cell death, which occurs normally in tissue remodeling, is generally considered immunologically silent, or even immunosuppressive. A possible exception is when apoptosis is accompanied by viral infection or other forms of stress. In contrast, necrotic cell death associated with infection and with other forms of stress is considered immune-stimulatory, giving rise to strong immune responses, notably class I-restricted cross-priming and the promotion of Th1 cells.

Irradiated cells classically die by apoptosis and therefore the use of irradiated whole tumor cells as vaccines may not appear ideal. However, vaccines will typically contain cell numbers in excess of what can be disposed of by scavenging macrophages and so vaccine cells, especially those undergoing secondary necrosis], will be able to provide a danger signal. Thus, antigen will be taken up by DC for priming of T cells. Alternatively, this signal could be provided by immunological adjuvants. In further support of cell-based vaccines, recent data suggest that cell-associated antigen is cross-presented to CD8+ T cells 50,000 times more efficiently than soluble antigen.

Difficulties in producing personalized whole tumor cell vaccines for every patient have led to the development of cross-reactive allogeneic (MHC-disparate) cell vaccines. The use of allogeneic cell vaccines separates the immune response into two phases because different tumor cells are present during the priming and effector stages. Studies have shown that in a murine melanoma model, vaccination of B6 mice with allogeneic K1735 melanoma cells provides significant protection against challenge with syngeneic B16 melanoma. This protection could not be improved upon by cytokine transfection of the vaccine cells or certain other adjuvants. This contrasted to K1735 in its syngeneic mouse (C3H) model, where the vaccine gave no protection from autologous challenge unless transfected with GM-CSF. Thus, although K1735 is not immunogenic as an autologous vaccine, it is relatively immunogenic as an allogeneic vaccine.

Allogeneic tumor cells as vaccines may be advantageous because the allogeneic molecules themselves providing immune stimuli which are capable of enhancing the immune response. This is due to a high proportion of host T cells that cross-react with allogeneic molecules (allo-recognition) leading to a reaction similar to that of host-versus-graft. Thus, an enhanced immunostimulatory environment within the vaccination site and secondary lymphoid tissue is generated. The induction of the chemokine MCP-1 by the B6 splenocytes may promote further APC infiltration into the vaccine site, whilst the tumor necrosis factor-alpha (TNF-) and IL-12 generated have the potential to induce APC maturation and enhance cell-mediated immunity. Spleen cells from K1735-vaccinated mice responded to K1735 by producing IFN-, demonstrating a Th1 recall response. Furthermore, allogeneic tumor cells induce an inflammatory cellular infiltrate at the site of injection in vivo. For example, subcutaneous injection of B6 mice with K1735 cells resulted in trafficking of cells with an APC-like surface phenotype (MHC class II+, CD80+, CD86+) into the injection site (manuscript in preparation). Thus, allogeneic molecules appear to provide an immune stimulatory signal, and indeed a number of studies have utilized such responses against cancer, for instance by transfecting tumor cells in situ with genes encoding allogeneic MHC molecules.

It has also been proposed that allogeneic APC may be able to prime T cells to recognize antigens on autologous tumors, or aid in this process. However, the majority of human cell vaccines in clinical use are transfected with GM-CSF to enhance tumor antigen cross-priming, whether the cells are of autologous or allogeneic origin

In brief, due to the immune system's inherent tolerance to many tumor antigens, additional immunological stimuli (e.g. GM-CSF production) are required to overcome this barrier. This additional stimulation may be in the form of an immunological adjuvant, or the direct removal or circumvention of specific regulatory constraints intrinsic to the immune system. However, it appears that one must exercise caution and strike a balance between immune stimulation and potential damage to healthy tissue. These immune constraints may be an even bigger challenge in humans because we are designed to prevent complex autoimmune responses, a by-product of the need to fight sophisticated pathogen challenges. Thus, although it appears that whole tumor cells may be a viable and practical human cancer vaccine, the overall protocol may need further immune modulation in order to maximize the potential therapeutic benefit to the patient.

4.3.1 DNA Vaccines and Associated Delivery Systems

The invention further provides means for introducing the a tumor antigen-encoding DNA into a subject so as to raise a T-cell mediated immune response. Various such DNA vaccine delivery systems are known in the art and exemplified below. Approaches to vaccination have developed rapidly (see e.g. Poland et al. (2002) BMJ 324: 1315-19 for review). DNA-based vaccination provides for protective immune responses by directly injecting engineered sequences from a desired target antigen (e.g. a tumor-specific antigen such as tumor antigen). The antigen is inserted into an expression vector (e.g. a poxvirus or an alphavirus-based vector. Once delivered into the host, the inserted DNA may undergo limited replication and the protein of interest is produced so that the host develops an immune response against the protein. In its simplest form, naked DNA (e.g. sequence of DNA inserted into bacterial plasmids and injected directly into the host to produce an immune response. Such naked DNA vaccines may be injected intramuscularly into human muscle tissue, or through transdermal or intradermal delivery of the vaccine DNA. Transdermally delivered microscopic gold beads coated with DNA encoding hepatitis B surface antigen generated protective immune responses—including the generation of CD8 cytotoxic lymphocytes (see Poland et al. (2001) Fourth annual Conference on Vaccine Research, Arlington, Va., April 23-25: S37: 57) (www.nfid.org/conferences/vaccine01-/abstracts/abss 37-40.pdf).

The invention provides these as well as numerous other DNA vaccine delivery systems known in the art and as exemplified below. Injection of “naked” plasmid DNA (PDNA) encoding Ag results in long-lasting cellular and humoral immune responses to Ag (Wolff et al. (1992) Hum. Mol. Genet. 1: 363). As described above, successful immunization has been demonstrated with administration of plasmid DNA by intramuscular, intradermal, intravenous, and subcutaneous routes. It has been reproducibly demonstrated that intramuscular injection of plasmid DNA provoke long-term immune responses characterized by the synthesis of specific IgG Abs, and by the efficient generation of CD8+ cytotoxic T cells and CD4+ Th1 cells (see Pardoll and Beckerleg (1995) Immunity 3: 165). For example, recent results have also indicated that plasmid DNA persists episomally without replication or incorporation into the host cell genome. Using intramuscular gene delivery, Hsu et al (Hsu et al. (1996) Nature Med 2: 540) have recently demonstrated that intramuscular injection of rats and mice with a pDNA encoding a house dust mite allergen (Der p 5) prevent the induction of IgE synthesis, histamine release, and airway hyper responsiveness in animals challenged with aerosolized allergen. Raz et al. (Raz et al. (1996) PNAS USA 93: 5141) showed that .beta.-galactosidase (.beta.-gal)/alum-primed Balb/c mice immunized intradermally with pDNA encoding .beta.-gal show a 66-75% reduction in the level of .beta.-gal-specific IgE in 6 weeks. Also this plasmid DNA immunization protocol induced specific IgG2a, and IFN-.gamma. secretion by the Th cells in the .beta.-gal/alum-primed mice. However, despite the recent success of DNA-based immunization in altering the IgE- and Th2-associated immune response in various models, the prophylactic and/or therapeutic potentials are far from clear. In the particular case of DNA vaccines directed against cancerous tumors for example, it is unpredictable whether the targeted antigen, even if tumor specific and available to immune surveillance, will be effective in producing the desired anti-tumor therapeutic effect.

Gene therapy vectors may be adapted for use in the instant invention. Recent clinical trials indicate that an efficient and safe delivery vehicle can be accomplished. Viral and retroviral vectors have been the most efficient and commonly used delivery modalities for in vivo gene transfer (see e.g. Xiang et al. (1996) Virology 219: 220 and below). However, Non-viral delivery systems are also included in the invention. Such systems may have potential advantages such as ease of synthesis, cell/tissue targeting, low immune response, and unrestricted plasmid size.

One promising non-viral gene delivery system thus far, other than the “gene gun” in DNA vaccine applications, comprises ionic complexes formed between DNA and polycationic liposomes (see e.g. Caplen et al. (1995) Nature Med. 1: 39). Held together by electrostatic interaction, these complexes may dissociate because of the charge screening effect of the polyelectrolytes in the biological fluid. A strongly basic lipid composition can stabilize the complex, but such lipids may be cytotoxic.

Complex coacervation is a process of spontaneous phase separation that occurs when two oppositely charged polyelectrolytes are mixed in an aqueous solution. The electrostatic interaction between the two species of macromolecules results in the separation of a coacervate (polymer-rich phase) from the supernatant (polymer-poor phase). This phenomenon can be used to form microspheres and encapsulate a variety of compounds. The encapsulation process can be performed entirely in aqueous solution and at low temperatures, and has a good chance, therefore, of preserving the bioactivity of the encapsulant. In developing an injectable controlled release system, the complex coacervation of gelatin and chondroitin sulfate to encapsulate a number of drugs and proteins has been exploited (see Truong, et al. (1995) Drug Delivery 2: 166) and cytokines have been encapsulated in these microspheres for cancer vaccination (see Golumbek et al. (1993) Cancer Res 53: 5841). Anti-inflammatory drugs have also been incorporated for intra-articular delivery to the joints for treating osteoarthritis (Brown et al. (1994) 331: 290). U.S. Pat. Nos. 6,193,970, 5,861,159 and 5,759,582, describe compositions and methods of use of complex coacervates for use as DNA vaccine delivery systems of the instant invention. In particular, U.S. Pat. No. 6,475,995, the contents of which are incorporated herein by reference, teaches DNA vaccine delivery systems utilizing nanoparticle coacervates of nucleic acids and polycations which serve as effective vaccines when administered orally. This oral DNA vaccine delivery system provides particularly preferred embodiments of the invention.

Other vaccine delivery systems are known in the art and/or described in U.S. Pat. Nos. 6,270,795; 6,294,378; 6,339,068; 6,358,933; 6,468,984; 6,472,375; 6,488,926; and 6,500,432; the contents of which are incorporated herein by reference.

4.4.1. Bacterial-Mediated DNA Vaccine Delivery Systems

In particularly preferred embodiments, the invention provides microorganism (e.g. bacterial)-based delivery systems for the DNA encoding the tumor antigen or other tumor antigen to be targeted by the DNA cancer vaccine. The use of live bacterial DNA vaccine vectors for antigen delivery has been reviewed recently (Medina and Guzman (2001) Vaccine 19: 1573-1580; Weiss and Chakraborty (2001) Current Opinion in Biotechnology 12: 467-72; and Darji et al. (2000) FEMS Immunol and Medical Microbiology 27: 341-9).

The use of live bacterial vaccine vectors is known in the art and described further herein. Furthermore, U.S. Pat. Nos. 6,261,568 and 6,488,926, the contents of which are incorporated herein by reference, describe particularly useful systems for use in the instant DNA cancer vaccine invention.

Significantly, the use of live bacterial vaccine vectors can be particularly advantageous. Bacteria-mediated gene transfer finds particular advantage in genetic vaccination by intramuscular, intradermal or oral administration of plasmids which leads to antigen expression in the mammalian host—thereby offering the possibility of both antigen modification as well as immune modulation. Furthermore, the bacterial-mediated DNA vaccine provides adjuvant effects and the ability to target inductive sites of the immune system. In preferred embodiments, S. typhimurium, S. typhi, S. flexneri or L. monocytogenes are used as vehicles for transkingdom DNA vaccine delivery.

Furthermore, live vaccine vectors make use of the almost unlimited coding capacity of bacterial plasmids, and broad availability of bacterial expression vectors, to express virtually any target tumor antigen of interest. The use of bacterial carriers is associated with still other significant benefits, such as the availability of convenient direct mucosal delivery. Other direct mucosal delivery systems (besides live viral or bacterial vaccine carriers) include mucosal adjuvants, viral particles, ISCOMs, liposomes, microparticles and transgenic plants. Other advantages of this technology are: low batch preparation costs, facilitated technology transfer following development of the prototype, increased shelf-life and stability in the field respect to other formulations (e.g. subunit vaccines), easy administration and low delivery costs. Taken together, these advantages make this strategy particularly suitable for DNA vaccine programs including cancer DNA vaccines. The carrier operationally becomes an equivalent of a subunit recombinant vaccine. This may in turn facilitate the critical evaluation of antigen-related side effects during clinical phases, when well-characterized carriers are used.

Both attenuated and commensal microorganisms have been successfully used as carriers for vaccine antigens. Attenuated mucosal pathogens which may be used in the invention include: L. monocytogenes, Salmonella spp., V. cholorae, Shigella spp., mycobacterium, Y. enterocolitica, and B. anthracis. Commensal strains for use in the invention include: S. gordonii, Lactobacillus spp., and Staphylococcus ssp. The background of the carrier strain used in the formulation, the type of mutation selected to achieve attenuation, and the intrinsic properties of the immunogen can be used in optimizing the extent and quality of the immune response elicited. The general factors to be considered to optimize the immune response stimulated by the bacterial carrier include carrier-related factors including: selection of the carrier; the specific background strain, the attenuating mutation and the level of attenuation; the stabilization of the attenuated phenotype and the establishment of the optimal dosage. Other considerations include antigen-related factors such as: intrinsic properties of the antigen; the expression system, antigen-display form and stabilization of the recombinant phenotype; co-expression of modulating molecules and vaccination schedules. The following bacterial vaccine vector delivery systems for use in the invention are reviewed in brief.

Listeria monocytogenes

In addition to being a preferred agent for the production of a tissue tropic immune response (e.g. in the liver for the treatment of hepatic tumors), Listeria monocytogenes may be used as a delivery for a DNA tumor/cancer vaccine of the invention. The Gram-positive bacterium L. monocytogenes invades phagocytic and non-phagocytic cells from a wide spectrum of animals, including humans, and escapes following internalization from the vacuole into the cytosol of the host cell. In the cytosol, it becomes motile by recruiting components of the host cell cytoskeleton and subsequently spreads to neighboring cells.

At present, a paucity of auxotrophic strains of L. monocytogenes that maintain the ability to efficiently invade host cells are available. A strain has been engineered to contain an autolysin that is activated intracellularly (Dietrich et al. (1998) Nature Biotechnol 16: 181-5). Using these bacteria, transfer of several reporter genes or cDNAs into a murine macrophage cell line could be shown. In addition, transfer into primary human dendritic cells was demonstrated. For in vivo transfer, bacteria carrying a GFP-encoding plasmid were injected into the peritoneum of mice and cotton rats. Cells harvested from these animals after a few days yielded macrophages that expressed the reporter gene.

Antibiotics may be used to achieve expression plasmid transfer from L. monocytogenes to host cells (i.e. after an appropriate infection time antibiotics were added to the cultures to kill intracellular bacteria). Several cell types of epithelial and endothelial origin from various species were tested successfully in these experiments (see Hense et al. (2001) Cell Microbiol 2001 3: 599-609. With some cell lines transfer to more then 10% of cells could be achieved. Invasion of the host cell and escape of the recombinant bacteria from the phagosome was essential for efficient plasmid transfer.

Stable transfectants in which the plasmids were integrated into the genome of the host cell were established using these transfer systems. Integration rates (i.e. how many stable clones could be derived from transiently transfected cells) ranged from 10-7 to 10-2 (see Dietrich et al. (1998) Nature Biotechnol 16: 181-5 and Hense et al. (2001) 3: 599-609). Although the reason for this wide range of integration rates is unclear it might represent a potential safety problem. Use of episomal vectors might provide a solution to this problem and may even improve the efficiency of transfer.

Recombinant Escherichia coli

Surprisingly, laboratory strains of E. coli K12 can also be used as transfer vectors for mammalian cells. Auxotrophic dapB mutants were used as previously described for S. flexneri. Because wild-type E. coli K12 is not invasive, it was transformed with the virulence plasmid of S. flexneri. This plasmid not only enabled mammalian cells of epithelial origin to be infected, but also facilitated subsequent escape from the phagosome. This E. coli was capable of transferring DNA to the eukaryotic cell (see Courvalin et al. (1995) 318: 1207-12). Additionally, E. coli have been generated that express the invasin gene of Yersinia pseudotuberculosis. Although these bacteria were also capable of invading host cells they were unable to egress from the vacuole; nevertheless, transfer of expression plasmids occurred (see Grillot-Courvalin (1998) Nat Biotechnol 16: 862-66). Such bacteria that expressed invasin together with an intracellular listeriolysin only showed moderately increased transfer rates at low multiplicity of infection (MOI). Little, if any, difference was detected in transfer rates with high MOIs between bacteria harboring invasin alone or invasin and listeriolysin. This indicates that the laboratory E. coli K12 has the ability to generate a port for transfer of expression plasmids across the phagosomal membrane into the nucleus of the host cell.

4.4 Tropic Inflammatory Agents

In general, the invention provides for immunostimulatory agents (e.g. microbes such as infectious bacteria, viruses and fungus) that possess a tropism for the organ or tissue to be targeted (e.g. the organ or tissue affected by cancer in the case of cancer therapeutics and associated methods of the invention). An infectious agent such as a virus, bacterium, yeast, or fungus with a natural tropism for a specific organ or tissue is suitable for use in this aspect of the invention. Furthermore, any infectious agent that can be engineered to localize to a specific organ or tissue (e.g. by fusion of a ligand for a receptor present on the organ or tissue onto an envelope, coat or membrane protein of the organism or by surface expression of or conjugation to an antibody specific to the organ or tissue) can be utilized. Methods for engineering organisms (e.g. bacteria and viruses) for tissue tropism are known in the art and described in, e.g., U.S. Pat. Nos. 6,514,722; 6,475,482; 6,472,368; 6,462,070; 6,440,419; 6,428,788; 6,428,771, 6,416,960; 6,410,517; 6,399,575; 6,379,699; 6,339,070; 6,331,524; 6,329,501; 6,261,787; 6,261,544; 6,252,058; 6,251,392; 6,221,647; 6,214,622; 6,080,849; 6,071,890; 6,004,554; 5,965,132; 5,863,538; 5,855,866; 5,851,527; 5,820,859; 5,776,427; and 5,660,827, the contents of which are incorporated herein in their entirety.

In particularly preferred applications, the tropic organism possesses or is engineered to possess tropism for the neovascular endothelium present in a developing tumor mass.

In preferred embodiments, the tropic agent, e.g. bacterial or viral or fungal organism, is further genetically engineered to produce chemokines, cytokines, adhesion molecules or other activators of immunity or inflammation (see e.g. section 4.6) by standard cloning methods known in the art (e.g. see Sanbrook et al. (1989) Molecular Cloning: A Laboratory Manual, 2^{nd} Edition, Cold Spring Harbor Press).

Other means for establishing tropism (e.g. by placement of the organism into a particular organ or tissue by physical means such as direct injection, percutaneous catheter, surgery or closed loop perfusion where the agent possesses no natural or engineered tropism are also contemplated in the method of the invention and discussed further below (see sections 4.8 and 4.9). For example, the agent may be localized to target the lungs by inhalation delivery and/or the gastrointestinal tract by ingestion.

4.4.1 Tropic Bacteria

Bacteria possessing a natural tropism for one or more tissue or organ are known in the art and include, without limitation, the following classes: those bacteria known to affect blood (i.e. bacteremia) including coagulase-negative staphylococci, Staphylococcus aureus Streptococcus pneumoniae, other Streptococcus species, Enterococcus, Escherichia coli, Klebsiella pneumoniae, Enterobacter, Proteus mirabilis, other Enterobacterioceae, Pseudomonas aeruginosa, other Pseudomonas species, Haemophilus influenzae, Bacteroides fragilis. Other bacteria known to affect blood include many aerobic and anaerobic bacteria.

Examples of bacteria that affect the heart (endocarditis) include Viridans group streptococci, Enterococcus, Staphylococcus aureus, Pseudomonas. Other bacteria known to affect the heart include Streptococcus pneumoniae, HACEK group (Haemophilus aphrophilus, Actinobacillus, Cardiobacterium, Eikenella, Kingella), Coxiella burnetii, Chlamydia psittaci.

Examples of bacteria that affect the Prosthetic valve include Coagulase-negative Staphylococci, Staphylococcus aureus, Enterococcus, Corynebacterium species. Other bacteria known to affect the valve are Streptococcus pneumoniae, Mycobacterium chelonae.

Examples of bacteria that affect the Central Nervous System (acute meningitis) include Streptococcus pneumoniae, Neisseria meningitidis, Haemophilus influenzae, group B Streptococcus, Listeria monocytogenes, Escherichia coli. Other bacteria known to affect the Central Nervous System are Leptospira, Staphylococcus aureaus. Examples of bacteria that affect chronic meningitis include Mycobacterium tuberculosis, Nocardia, Treponema pallidum. Other bacteria known to affect chronic meningitis include Borrelia burgdorferi, Brucella, and other mycobacterial species.

Examples of bacteria that affect Brain abscess include Viridans group streptococci, mixed anaerobes (Bacteroides, Fusobacterium, Porphyromonas, Prevotella, Peptostreptococcus), Staphylococcus aureus. Other bacteria known to affect Brain abscess are Clostridium species, Haemophilus, Nocardia, and Enterobacteriaceae.

Examples of bacteria that affect Intra-abdominal infection (spontaneous peritonitis) are Escherichia coli, Klebsiella pneumoniae, Streptococcus pneumoniae, Enterococcus. Other bacteria known to affect Intraabdominal infection are Staphylococcus aureaus, anaerobes, Neisseria gonorrhoeae, Chlamydia trachomatis, Mycobacterium tuberculosis.

Examples of bacteria that affect the secondary peritonitis are Escherichia coli, Bacteroides fragilis, other enteric anaerobes, Enterococcus, Pseudomonas aeruginosa. Other bacteria known to affect Secondary peritonitis are Staphylococcus aureus, Neisseria gonorrhoeae, Mycobacterium tuberculosis.

Examples of bacteria that affect the dialysis-associated peritonitis are Coagulase-negative Staphylococcus, Staphylococcus aureus, Streptococcus species, Corynebacterium species. Other bacteria known to affect Dialysis-associated peritonitis are Escherichia coli, Klebsiella, Enterobacter, Proteus, Pseudomonas.

Examples of bacteria that affect the intraabdominal abscess are Bacteroides fragilis group, Escherichia coli, Enterococcus. Other bacteria known to affect Intraabdominal abscess are Klebsiella, Enterobacter, Proteus, Pseudomonas, Staphylococcus aureus.

Examples of bacteria that affect the upper respiratory tract (Pharyngitis) are Group A Streptococcus. Other bacteria known to affect upper respiratory tract are mixed anaerobes (Vincent's angina), Neisseria gonorrhoeae, Corynebacterium diphtheriae, Corynebacterium ulcerans, Archanobacterium haemolyticum, Mycoplasma pneumoniae, Yersinia enterocolitica.

Examples of bacteria that affect the tracheobranchitis include M. Pneumoniae.

Examples of bacteria that affect the otitis externa are Streptococcus pneumoniae, Haemophilus influenzae, Moraxella catarrhalis, anaerobes. Other bacteria known to affect otitis externa/media are Staphylococcus aureus, group A Streptococcus.

Examples of bacteria that affect Sinusitis are Streptococcus pneumoniae, Haemophilus influenzae, Moraxella catarrhalis, anaerobes. Other bacteria known to affect Sinusitis are Streptococcus aureus, Group A Streptococcus.

Examples of bacteria that affect the epiglottis are Haemophilus influenzae. Other bacteria known to affect the epiglottis are Streptococcus pneumoniae, Staphylococcus aureus, other Haemophilus species.

Examples of bacteria that affect the lower respiratory tract (Bronchitis) are Mycoplasma pneumoniae, Bordetella pertussis, Chlamydia species.

Examples of bacteria that affect acute pneumonia are Streptococcus pneumoniae, Staphylococcus aureus, Haemophilus influenzae, Klebsiella pneumoniae, Escherichia coli, Legionella, Pseudomonas aeruginosa, mixed anaerobes, Mycoplasma pneumoniae, Chlamydia. Other bacterial known to affect acute pneumonia are Acinetobacter, Moraxella catarrhalis, Neisseria meningitidis, Mycobacterium tuberculosis, other Mycobacterium species, Eikenella, Francisella, Nocardia, Pasteurella multocida, Pseudomonas pseudomallei, Yersinia pestis, Coxiella burnetii, Rickettsia, Bacillus anthracis.

Examples of bacteria that affect chronic pneumonia are mixes anaerobes, Mycobacterium tuberculosis, Nocardia. Other bacteria known to affect chronic pneumonia are Actinomyces, Pseudomonas pseudomallei, Mycobacterium species.

Examples of bacteria that affect the Eye (conjunctivitis) are Streptococcus pneumoniae, Staphylococcus aureus, coagulase-negative staphylococci, Haemophilus influenzae (H. aegyptius), Neisseria gonorrhoeae, Chlamydia trachomatis.

Examples of bacteria that affect Keratitis are Staphylococcus aureus, Streptococcus pneumoniae, Pseudomonas aeruginosa, Moraxella. Other bacteria known to affect Keratitis are Mycobacterium fortuitum-chelonae.

Examples of bacteria that affect Endophthalmitis are Staphylococcus aureus, Pseudomonas aeruginosa, Bacillus species.

Examples of bacteria that affect Skin and soft tissue infections: Impetigo—Group A Streptococcus, Staphylococcus aureus; Furuncles and carbuncles—Staphylococcus aureus; Paronychia—Staphylococcus aureus, Group A Streptococcus, Pseudomonas aeruginosa; Erysipelas—Group A Streptococcus; Cellulitis—Group A Streptococcus, Staphylococcus aureus, Haemophilus influenzae; Necrotizing cellulitis and fascitis—Group A Streptococcus, Clostridium perfringens, other clostridial species, Bacteroides fragilis, other gram-negative anaerobes, Peptostreptococcus, Enterobacteriaceae, Pseudomonas aeruginosa; Chancriform lesions—Treponema pallidum, Haemophilus ducreyi. Other bacteria known to affect Chancriform lesions are Bacillus anthracis, Francisella tularensis, Mycobacterium ulcerans, Mycobacterium marinum; Wounds caused by trauma, burns, bites, etc. includes a large variety of organisms including staphylococci, streptococci, Enterobacteriaceae, Pseudomondaceae, and other environmental bacteria

Examples of bacteria that affect the bone and joint in arthritis include—Staphylococcus aureus, Neisseria gonorrhoeae, Streptococcus species, Haemophilus influenzae. Other bacteria known to affect Arthritis are Brucella, Nocardia, Mycobacterium species. Osteomyelitis—Staphylococcus aureaus, Enterobacteriaceae (Salmonella, Escherichia, Klebsiella, Proteus), Pseudomonas. Other bacteria known to affect Osteomyellitis are Mycobacterium tuberculosis, other mycobacterial species, anaerobes. Prosthesis-associated infections include Staphylococcus aureus, coagulase-negative staphylococci, Streptococcus species. Other bacteria known to affect Prosthesis-associated infections

Peptostreptococcus, miscellaneous aerobic gram-negative bacilli.

Examples of bacteria that affect the urinary tract include: cystitis-causing organisms such as Escherichia coli, Proteus mirabilis, Klebsiella, Enterobacter, Pseudomonas, Enterococcus, Staphylococcus saprophyticus. Other bacteria known to affect cystitis are Staphylococcus aureus, Corynebacterium ureolyticus, Clostridium species, Bacteroides fragilis, Ureaplasma urealyticum; Pyelonephritis—Escherichia coli, Proteus mirabilis, Klebsiella, Staphylococcus aureus. Other bacteria known to affect Pyelonephritis are Enterococcus, Corynebacterium ureolyticus. Prostatitis—Escherichia coli, Klebsiella, Enterobacter, Proteus mirabilis, Enterococcus. Other bacteria known to affect Prostatitis are Neisseria gonorrhoeae.

Examples of bacteria that affect the genitals include those which cause urethritis—Neisseria gonorrhoeae, Chlamydia trachomatis. Other bacteria known to affect Genital Urethritis are Ureaplasma urealyticum, Mycoplasm genitalum. Bacterial vaginosis (vaginitis), synergistic infection with anaerobes (e.g., Mobiluncus, Bacteroides species, Peptostreptococcus) and possibly Gardnerella vaginalis. Cervicitis—Neisseria gonorrhoeae, Chlamydia trachomatis. Other bacteria known to affect cervicitis are Actinomyces, Mycobacterium tuberculosi. Genital ulcers—Treponema pallidum, Haemophilus ducreyi, Chlamydia trachomatis (LGV). Other bacteria known to affect Genital ulcers are Actinomyces, Mycobacterium tuberculosis

Examples of bacteria that affect Gastrointestinal Intoxication (disease caused by toxin in food): Staphylococcus aureus, Bacillus cereus, Clostridium botulinum. Infection—Camphylobacter, Salmonella, Shigella, Clostridium difficile, Clostridium perfringens, Clostridium botulinum (infant botulism), Vibrio cholerae, Vibrio parahaemolyticus, Bacillus cereus. Other bacteria known to affect Infection are Escherichia coli (enterotoxigenic, enteroinvasive, enteropathogenic, enterohemorrhagic), other toxin-producing Enterobacteriaceae), Aeromonas, Plesiomonas, Yersinia enterocolitica. Gastritis—Heliobacter. Proctitis—Neisseria gonorrhoeae, Chlamydia trachomatis, Treponema pallidum.

Listeria monocytogenes

In a particularly preferred example of the invention, intraperitoneal injection of Listeria monocytogenes (preferably one attenuated with HIV-gag; see Lieberman and Frankel (2002) Vaccine 20: 2007-10; and Friedman et al. (2000) J Virol 74: 9987-93) results in tropic localization of this bacterium to the liver (e.g. for use in augmenting a liver tumor vaccine for the treatment of liver cancer).

It is known that most bacteria that enter the bloodstream, including Listeria, are taken up and eliminated with the liver via systemic uptake mechanisms (see Gregory and Wing (2002) J Leukoc Biol 72: 239-48 for review). The pathophysiology of Listeria infection in humans generally occurs through contaminated food in both epidemic and sporadic cases—the gastrointestinal tract is thought to be the primary site of entry of pathogenic Listeria organisms into the host (see Vazquez-Boland et al. (2001) Clin Microbiol Rev 14: 584-640 for review). The clinical course of infection usually begins about 20 h after the ingestion of heavily contaminated food in cases of gastroenteritis, whereas the incubation period for the invasive illness is generally much longer, around 20 to 30 days. Similar incubation periods have been reported in animals for both gastroenteric and invasive disease.

Human immunodeficiency virus (HIV) infection is also a significant risk factor for listeriosis. AIDS is the underlying predisposing condition in 5 to 20% of listeriosis cases in nonpregnant adults. It has been estimated that the risk of contracting listeriosis is 300 to 1,000 times higher for AIDS patients than for the general population. Nevertheless, listeriosis remains a relatively rare AIDS-associated infection, probably due to the preventive dietary measures taken by HIV-infected patients (avoidance of high-risk foods), the antimicrobial treatments that they receive regularly to treat or prevent opportunistic infections, and the fact that HIV infection does not significantly reduce the activity of the major effectors of immunity of Listeria spp. (innate immune mechanisms and the CD8+ T-cell subset.

Entry and colonization of host tissues by Listeria generally occurs by crossing the intestinal barrier. Before reaching the intestine, the ingested Listeria organisms must withstand the adverse environment of the stomach. Oral infective doses are lower for cimetidine-treated experimental animals than for untreated animals, and the use of antacids and H2-blocking agents has been reported to be a risk factor for listeriosis. This indicates that gastric acidity may destroy a significant number of the Listeria organisms ingested with contaminated food.

Listeria multiplies in the liver. The Listeria organisms that cross the intestinal barrier are carried by the lymph or blood to the mesenteric lymph nodes, the spleen, and the liver. This initial step of host tissue colonization by L. monocytogenes is rapid. The unusually long incubation period required by L. monocytogenes for the development of symptomatic systemic infection after oral exposure in relation to that for other food-borne pathogens is therefore puzzling and indicates that listerial colonization of host tissues involves a silent, subclinical phase, many of the events and underlying mechanisms of which are unknown.

Experimental infections of mice via the intravenous route have shown that L. monocytogenes bacteria are rapidly cleared from the bloodstream by resident macrophages in the spleen and liver. Most (90%) of the bacterial load accumulates in the liver, presumably captured by the Kupffer cells that line the sinusoids. These resident macrophages kill most of the ingested bacteria, as shown by in vivo depletion experiments, resulting in a decrease in the size of the viable bacterial population in the liver during the first 6 h after infection. Kupffer cells are believed to initiate the development of antilisterial immunity by inducing the antigen-dependent proliferation of T lymphocytes and the secretion of cytokines. Not all Listeria cells are destroyed by tissue macrophages, and the surviving bacteria start to grow, increasing in numbers for 2 to 5 days in mouse organs.

The principal site of bacterial multiplication in the liver is the hepatocyte. This finding has led to the dismissal of the long-held idea that the major host niche for the parasitic life of L. monocytogenes is the macrophage population. There are two possible ways for L. monocytogenes to gain access to the liver parenchyma after its intestinal translocation and carriage by the portal or arterial bloodstream: via Kupffer cells, by cell to cell spread, or by the direct invasion of hepatocytes from the Disse space after crossing the fenestrated endothelial barrier lining the sinusoids. Indeed, L. monocytogenes has been shown to efficiently invade hepatocytes in vitro.

Electron microscopy of hepatic tissue from infected mice suggests that L. monocytogenes goes through the complete intracellular infectious cycle in hepatocytes, including actin-based intercellular spread. Direct passage from hepatocyte to hepatocyte would lead to the formation of infectious foci in which L. monocytogenes disseminates through the liver parenchyma without coming into contact with the humoral effectors of the immune system. This may explain why antibodies play no major role in anti-Listeria immunity.

Listeria may also colonize the gravid uterus and fetus. Abortion and stillbirth due to Listeria spp. have been reproduced experimentally by intravenous, oral, and respiratory inoculation in naturally susceptible gestating animal hosts, such as sheep, cattle, rabbits, and guinea pigs, as well in pregnant mice and rats. This shows that L. monocytogenes gains access to the fetus by hematogenous penetration of the placental barrier. In pregnant mice, the blood-borne bacteria first invade the decidua basalis and then progress to the placental villi, where they cause diffuse inflammatory infiltration and necrosis. Macrophages appear to be excluded from the murine placenta, neutrophils acting as the main antilisterial effector cell population. Using homozygous mutant mice, it has been shown recently that colony-stimulating factor-1 is required for the recruitment of neutrophils to the infectious foci in the decidua basalis. This occurs via induction of neutrophil chemoattractant synthesis by the trophoblast. In humans, placental infection is characterized by numerous microabscesses and focal necrotizing villitis. Colonization of the trophoblast layer followed by translocation across the endothelial barrier would enable the bacteria to reach the fetal bloodstream, leading to generalized infection and subsequent death of the fetus in utero or to premature birth of a severely infected neonate with miliary pyogranulomatous lesions (the above-mentioned granulomatosis infantiseptica). The depression of cell-mediated immunity during pregnancy presumably plays an important role in the development of listeriosis.

Listeria is also capable of invasion of the brain. In humans, CNS infection by Listeria spp. presents primarily in the form of meningitis. This meningitis, however, is often associated with the presence of infectious foci in the brain parenchyma, especially in the brain stem, suggesting L. monocytogenes has a tropism for nerve tissue. The neurotropism and special predilection of L. monocytogenes for the rhombencephalon are shown most clearly in ruminants, in which listerial CNS infection, in contrast to the situation in humans, develops mainly as primary encephalitis. In these animals, infectious foci are restricted to the pons, medulla oblongata, and spinal cord. Although there is inflammatory lymphocyte or mononuclear cell infiltration of the meninges, this condition occurs as an extension of the brain process, and macroscopic lesions may not even be evident or may be restricted to basal areas, midbrain, and cerebellum. Unilateral cranial nerve paralysis is a characteristic of listerial rhombencephalitis in ruminants, leading to the well-known circling disease syndrome. In humans, primary nonmeningeal brain infection is seldom observed. However, as in ruminants, it develops as cerebritis involving the rhombencephalon.

Brain lesions in listerial meningoencephalitis are typical and very similar in humans and animals. They consist of perivascular cuffs of inflammatory infiltrates composed of mononuclear cells and scattered neutrophils and lymphocytes. Bacteria are generally absent from these perivascular areas of inflammation. Parenchymal microabscesses and foci of necrosis and malacia are also typically present. Bacteria are relatively abundant in these lesions, within phagocytes or free in the brain parenchyma around the necrotic areas. Depletion experiments in mice using a neutrophil-specific monoclonal antibody have shown that neutrophils play a critical role in eliminating L. monocytogenes from infectious foci in the brain. Less commonly, bacteria are observed within neurons in both natural and experimentally induced infections. This is consistent with in vitro data showing that the invasion of cultured neurons is a relatively rare event. However, neurons are efficiently invaded in vitro by direct cell-to-cell spread from infected macrophages or microgial cells

Attenuation of Listeria, in addition to the HIV-gag attenuated Listeria cited above, can be effected through inactivation of known virulence gene expression using methods known in the art (see Vazquez-Boland et al. (2001) Clin Microbiol Rev 14: 584-640 for review of Listeria virulence factors and gene organization and expression).

4.4.2 Tropic Viruses

Examples of tropic viruses include: Hepatitis A, B, C, D and E, yellow fever, and Epstein-Barr viruses, which infect the liver; Cytomegalovirus, Herpes simplex virus, Varicella and Rubella viruses, which infect the liver in neonates or immuno-compromised individuals; Coxsackie B virus, which infects the heart; Cytomegalovirus, which infects the kidney; Coxsackie B (pleurodynia) virus, which infects muscle; Cytomegalovirus and Mumps virus, which infect glands; Herpes simplex virus, Adenovirus, Measles, Rubella, Enterovirus 70 and Coxsackie A24 viruses, which infect the eye.

4.4.3. Other Tropic Agents

The invention further provides for other agents, including fungal and parasitic organisms and even “nonliving” inflammatory agents (including small molecules) that naturally or, via chemical engineering, target the desired tumor or organ.

Examples of fungi that infect specific tissues causing superficial mycoses include Malassezia furfur and Exophiala werneckii which infect the skin.

Examples of parasitic organisms that infect specific tissues and organs include: Leishmania spp. , which infect bone marrow; Acanthamoeba Naegleria, Trypanosomes and Angiostrongylus cantonensis, which infect the central nervous system, Leishmania spp., which infect the eye; Entamoeba histoytica, Giardia, Cryptospridium, Microsporidia, pinworm and helminths, which infect the intestinal tract; E. histolytica and Leishmania spp., which infect the liver and spleen; Pneumocystis carinii, which infect the lung; Trichinella spiralis and trypanosoma cruzi, which infect the muscle; onchocerca volvulus, and Leishmania spp., which infect the skin; and, finally, Trichomonas vaginalis and Schistosoma haematobium, which infect the urogenital system.

4.5. Nucleic Acids and Polypeptides

The invention provides tumor antigen-encoding and immunostimulatory-stimulatory factor-encoding (e.g. cytokines such as granulocyte-macrophage colony-stimulating factor (GM-CSF see GenBank No. NM_{—}000758 and U.S. Pat. No. 5,641,663, the contents of which are incorporated herein) and other nucleic acids, homologs thereof, and portions thereof, and the polypeptides they encode. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferably 95% and even more preferably at least 99% homologous with a nucleotide sequence of a subject gene, e.g., an tumor antigen-encoding gene Nucleic acids at least 90%, more preferably 95%, and most preferably at least about 98-99% identical with a nucleic sequence represented in one of the subject nucleic acids of the invention or complement thereof are of course also within the scope of the invention. In preferred embodiments, the nucleic acid is mammalian and in particularly preferred embodiments, includes all or a portion of the nucleotide sequence corresponding to the coding region which correspond to the coding sequences of the subject tumor antigen-encoding DNAs.

The invention also pertains to isolated nucleic acids comprising a nucleotide sequence encoding tumor antigen polypeptides, variants and/or equivalents of such nucleic acids. The term equivalent is understood to include nucleotide sequences encoding functionally equivalent tumor antigen polypeptides or functionally equivalent peptides having an activity of a tumor antigen protein such as described herein. Equivalent nucleotide sequences will include sequences that differ by one or more nucleotide substitution, addition or deletion, such as allelic variants; and will, therefore, include sequences that differ from the nucleotide sequences of e.g. the corresponding tumor antigen gene GenBank entries due to the degeneracy of the genetic code.

Preferred nucleic acids are vertebrate tumor antigen nucleic acids. Particularly preferred vertebrate tumor antigen nucleic acids are mammalian. Regardless of species, particularly preferred tumor antigen nucleic acids encode polypeptides that are at least 60%, 65%, 70%, 72%, 74%, 76%, 78%, 80%, 90%, or 95% similar or identical to an amino acid sequence of a vertebrate tumor antigen protein. In one embodiment, the nucleic acid is a cDNA encoding a polypeptide having at least one bio-activity of the subject tumor antigen polypeptides or APC-stimulatory factors. Preferably, the nucleic acid includes all or a portion of the nucleotide sequence corresponding to the nucleic acids available through GenBank.

Still other preferred nucleic acids of the present invention encode a tumor antigen-encoding polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

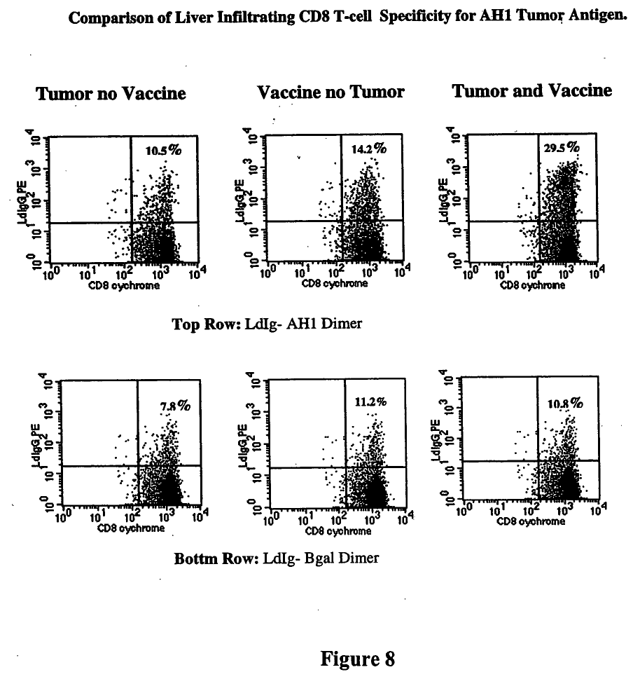

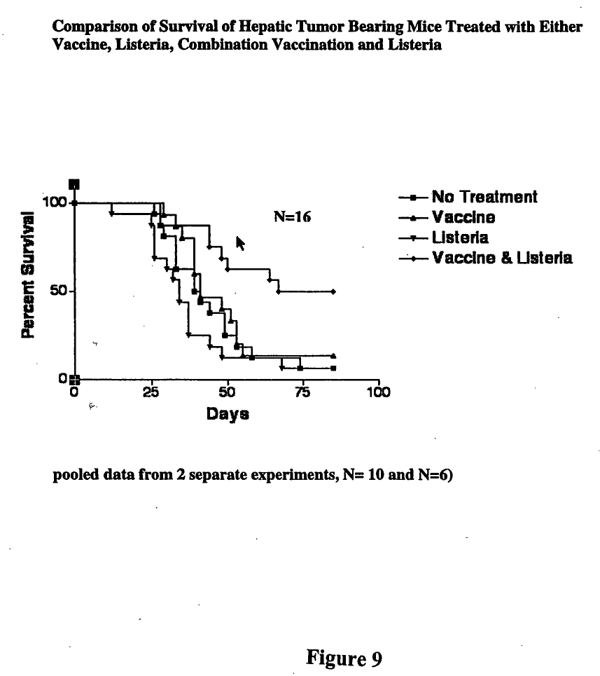

Another aspect of the invention provides a nucleic acid which hybridizes under stringent conditions to a nucleic acid represented by any of the subject nucleic acids of the invention. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an tumor antigen nucleic acid of the present invention will bind to one of the subject SEQ ID Nos. or complement thereof under moderately stringent conditions, for example at about 2.0×SSC and about 40° C. In a particularly preferred embodiment, a tumor antigen-encoding nucleic acid of the present invention will bind to one of the nucleic acid sequences of FIG. 8A or 9A or complement thereof under high stringency conditions. In another particularly preferred embodiment, a tumor antigen-encoding nucleic acid sequence of the present invention will bind to one of the nucleic acids of the invention which correspond to a tumor antigen-encoding ORF nucleic acid sequences, under high stringency conditions.

Nucleic acids having a sequence that differs from the nucleotide sequences shown in one of the nucleic acids of the invention or complement thereof due to degeneracy in the genetic code are also within the scope of the invention. Such nucleic acids encode functionally equivalent peptides (i.e., peptides having a biological activity of a tumor antigen-encoding polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a Tumor antigen polypeptide. However, it is expected that DNA sequence polymorphisms that do lead to changes in the amino acid sequences of the subject tumor antigen polypeptides will exist among mammals. One skilled in the art will appreciate that these variations in one or more nucleotides (e.g., up to about 3-5% of the nucleotides) of the nucleic acids encoding polypeptides having an activity of an tumor antigen-encoding polypeptide may exist among individuals of a given species due to natural allelic variation.

4.5.1 Probes and Primers

The nucleotide sequences determined from the cloning of tumor antigen genes from mammalian organisms will further allow for the generation of probes and primers designed for use in identifying and/or cloning other tumor antigen homologs in other cell types, e.g., from other tissues, as well as tumor antigen homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from one of the nucleic acids (e.g. an tumor antigen-encoding nucleic acid) of the invention.

In preferred embodiments, the tumor antigen primers are designed so as to optimize specificity and avoid secondary structures which affect the efficiency of priming. Optimized PCR primers of the present invention are designed so that “upstream” and “downstream” primers have approximately equal melting temperatures such as can be estimated using the formulae: Tₘ=81.5° C.−16.6(log₁₀[Na⁺])+0.41(% G+C)−0.63 (% formamide)−(600/length); or Tₘ(° C.)=2(A/T)+4(G/C). Optimized Tumor antigen primers may also be designed by using various programs, such as “Primer3” provided by the Whitehead Institute for Bi

Likewise, probes based on the subject tumor antigen sequences can be used to detect transcripts or genomic sequences encoding the same or homologous proteins, for use, e.g., in prognostic or diagnostic assays (further described below). The invention provides probes which are common to alternatively spliced variants of the tumor antigen transcript, such as those corresponding to at least 12 consecutive nucleotides complementary to a sequence found in any of the gene sequences of the invention. In addition, the invention provides probes which hybridize specifically to alternatively spliced forms of the tumor antigen transcript. Probes and primers can be prepared and modified, e.g., as previously described herein for other types of nucleic acids.

4.5.2. Antigens

The invention provides for antigens and tumor antigens and tumor antigen-expressing genes for use in the invention as described below.

Where the antigen encoded by the transduced expression vector is a pathogen antigen, such as a bacterial or viral tumor antigen, the invention allows for the treatment and protection against infectious disease—i.e. in traditional DNA vaccine applications. Numerous pathogen antigens for use in this aspect of the invention are known in the art and may be obtained using e.g. standard cloning techniques and/or the nucleic acid and polypeptide sequence information provided in GenBank and other sources (see e.g. www.ncbi.nlm.nih.gov/entrez).

Exemplary pathogen antigens for use in the invention include: hepatitis B tumor antigen (e.g. HBcAg or the secreted form HBeAg of the core protein of hepatitis B virus (HBV), see e.g. Kuhrober (1997) Int Immunol 9: 1203-12) for use in treating and preventing hepatitis B infection; tuberculosis antigen for use in treating and preventing tuberculosis (see e.g. Montgomery (2000) Brief Bioinform 1: 289-96); HIV tumor antigen (e.g. gp160) for use in treating and preventing HIV infections (see e.g. Schultz et al. (2000) Intervirology 43: 197-217); and Borrelia burgdorferi sensu lato antigens (e.g. outer surface lipoprotein A (OspA)) for treating and preventing Lyme disease (see e.g. Simon et al. (1999) Zentralbl Bakteriol 289: 690-5). Moreover, the sequencing of bacterial genomes and subsequent identification of surface-exposed microbial structures and their conservation in natural populations of pathogenic species allows for the rapid identification of prime candidates for many additional pathogen antigens for use in the invention (see e.g. Saunder and Moxon (1998) Curr Opin Biotechnol 9: 618-23).

Where the antigen encoded by the transduced expression vector is a tumor antigen, the invention allows for the treatment of cancers—e.g. metastatic hepatic tumors. Numerous tumor antigens for use in this aspect of the invention are known in the art and may be obtained using e.g. standard cloning techniques and/or the nucleic acid and polypeptide sequence information provided in GenBank and other sources (see e.g. www.ncbi.nlm.nih.gov/entrez).

Exemplary tumor antigens for use in the invention include: the prostate-specific membrane tumor antigen (PSMA) to treat prostate cancer (see e.g. Mincheff et al. (2000) Eur Urol 38: 208-17); the HER2/neu gene tumor antigen to treat breast cancer (see e.g. Lachman et al. (2001) Cancer Gene Ther 8: 259-68); idiotypic immunoglobulin sequences to treat B-cell malignancies (see e.g. Stevenson et al. (2001) Ann Hematol 80 suppl 3: B132-4); idiotypic T cell receptor tumor antigens to treat T cell malignancies (see e.g. Reddy et al. (2001) Ann NY Acad Sci 941: 97-105); an SV40 tumor antigen to treat SV40-expressing tumors (see e.g. Watts et al. (2000) Dev Biol (Basel) 104: 143-7); and carcinoembryonic tumor antigen (CEA) and CD40 ligand tumor antigen to treat carcinomas (see e.g. Xiang et al. (2001) J Immunol 167: 4560-5).

Also included are fusions of such tumor antigens to tumor antigenic polypeptides (e.g. tetanus toxin polypeptides see e.g. Stevenson et al. (2001) Ann Hematol 80 suppl 3: B132-4) to increase the immune response to the tumor antigen.

4.6. GM-CSF and Other Immunostimulatory Agents

In certain embodiments, e.g. in conjunction with a genetically engineered whole tumor cell vaccine, the invention provides for immunostimulatory agents for use in conjunction with the vaccine and tropic agents of the invention. Various cytokines and other molecules can stimulate the growth, differentiation, migration, and activation of dendritic cells or other tumor antigen presenting cells and can also boost the ability of dendritic cells to trigger and enhance T cell responses to tumor antigen presentation. See, e.g., Banchereau J et al., “Dendritic cells and the control of immunity.” Nature (1998) 392: 245-52; Young J W et al., “The hematopoietic development of dendritic cells: a distinct pathway for myeloid differentiation.” Stem Cells, (1996) 14:376-387; Cella M et al., “Origin, maturation and tumor antigen presenting function of dendritic cells.” Curr Opin Immunol. (1997) 9:10-16; Curti A et al., “Dendritic cell differentiation from hematopoietic CD34⁺ progenitor cells.” J. Biol. Regul. Homeost. Agents (2001) 15:49-52.

Examples of molecules that can modulate differentiation, maturation, expansion or activation of dendritic cells or other tumor antigen presenting cells include ligands such as CD40 ligand, granulocyte-macrophage colony stimulating factor (GM-CSF), FMS-like receptor tyrosine kinase 3 ligand (Flt3 ligand, FL), interleukin (IL) 1-alpha, IL 1-beta, IL-3, IL-4, IL-6, IL-12, IL-13, IL-15, tumor necrosis factor alpha (TNF-α), granulocyte colony stimulating factor (G-CSF), stem cell factor (SCF, also known as kit ligand, KL, Steel Factor, SF, SLF, and Mast cell growth factor, MGF), tumor necrosis factor (TNF)-related activation-induced cytokine (TRANCE), and tumor necrosis factor-related apoptosis-inducing ligand (TRAIL), and transforming growth factor β1. Fusion proteins having one or more activities ascribed to any of the above molecules may also modulate differentiation, maturation, expansion, or activation of dendritic cells or other tumor antigen presenting cells. Any of these ligands, fusion proteins, or other molecules could be encoded as a second gene expression cassette in a vector expression system.

CD40 ligand has been reported to promote induction of dendritic cells and facilitate development of immunogenic responses. See, e.g., Borges L et al., “Synergistic action of fms-like tyrosine kinase 3 ligand and CD40 ligand in the induction of dendritic cells and generation of antitumor immunity in vivo.” J Immunol. (1999) 163:1289-1297; Grewal I, Flavell R. “The CD40 ligand. At the center of the immune universe?” Immunol Res. (1997)16:59-70. Exemplary nucleic acids that encode CD40 ligand and equivalents are described (see, e.g. Genbank accession nos. X65453 and L07414), as are preparations, compositions, and methods of use (U.S. Pat. No. 6,290,972 to Armitage et al.)

GM-CSF (for exemplary nucleic acids encoding GM-CSF and equivalents, see, e.g., Genbank accession nos. X03020, X03019, X03221, E02975, E02287, E01817, E00951, E00950, A20083, A11763, and X03021) has been reported modulate mobilization, differentiation, expansion, and activation of dendritic cells and other tumor antigen presenting cells. See, e.g., Arpinati M et al., “Granulocyte-colony stimulating factor mobilizes T helper 2-inducing dendritic cells.” Blood. (2000) 95(8):2484-2490; Pulendran B et al., “Flt3-ligand and granulocyte colony-stimulating factor mobilize distinct human dendritic cell subsets in vivo.” J Immunol. (2000) 165(1):566-572; Sallusto F, Lanzavecchia A, “Efficient presentation of soluble tumor antigen by cultured human dendritic cells is maintained by granulocyte/macrophage colony-stimulating factor plus interleukin 4 and downregulated by tumor necrosis factor α.” J Exp Med (1994) 182: 389-400; Szabolcs P et al., “Expansion of immunostimulatory dendritic cells among the myeloid progeny of human CD34⁺ bone marrow precursors cultured with c-kit ligand, granulocyte-macrophage colony-stimulating factor, and TNF-α.” J Immunol (1995) 154: 5851-61; Caux C et al., “Tumor necrosis factor a strongly potentiates interleukin-3 and granulocyte-macrophage colony-stimulating factor-induced proliferation of human CD34⁺ hematopoietic progenitor cells.” Blood (1990) 75: 2292-8. Compositions, preparations, methods of manufacture and use, analogs, fusions, and equivalents of GM-CSF-encoding exemplary nucleic acid are described, e.g., in U.S. Pat. Nos. 5,641,663, 5,908,763, 5,891,429, 5,393,870, 5,073,627, 5,359,035, and in foreign patent documents JP 1991155798, JP 1990076596, JP 1989020097, GB 2212160, EP 0352707, EP 0228018, and WO8504188).

Flt3-ligand has been described to modulate mobilization, induction, and proliferation of dendritic and other tumor antigen presenting cells. See, e.g., Pulendran B et al., “Flt3-ligand and granulocyte colony-stimulating factor mobilize distinct human dendritic cell subsets in vivo.” J Immunol. (2000) 165(1):566-572; Borges L et al., “Synergistic action of fms-like tyrosine kinase 3 ligand and CD40 ligand in the induction of dendritic cells and generation of antitumor immunity in vivo.” J Immunol. (1999) 163:1289-1297; Lebsack M et al., “Safety of FLT3 ligand in healthy volunteers.” Blood (1997) 90(Suppl. 1, Abstract 751):170a; Lyman SD. Biologic effects and potential clinical applications of Flt3 ligand. Curr Opin Hematol. (1998) 5(3): 192-196; Maraskovsky E et al., “Dramatic increase in the numbers of functionally mature dendritic cells in FLT3-ligand-treated mice: multiple dendritic cell subpopulations identified.” J Exp Med (1996) 184:1953-62; Strobl H, et al., “Flt3-ligand in cooperation with transforming growth factor-β1 potentiates in vitro development of Langherans-type dendritic cells and allows single-cell dendritic cell cluster formation under serum-free conditions.” Blood (1997) 90:1425-34. Exemplary nucleic acids encoding Flt3 ligand and equivalents are disclosed, e.g., in Genbank accession nos. NM_{—}013520, L23636, U04807, U44024, U29875, U03858, U29874, and U04806). Preparations, compositions, and methods of use are described, e.g., in U.S. Pat. Nos. 6,291,661, 5,843,423, and 5,554,512.

Exemplary nucleic acids encoding IL-12 and equivalents are described, e.g., in Genbank accession nos. AF401989, AF411293, AF180563, AF180562, AF101062, AY008847, XM_{—}084136, M65271, AF050083, XM_{—}004011, M86672, NM_{—}008351, M86671, and NM_{—}008352 and in U.S. Pat. No. 5,723,127 to Scott et al.

TNF-α has been found to affect multiple aspects of dendritic cell proliferation and development. See, e.g., Szabolcs P et al., “Expansion of immunostimulatory dendritic cells among the myeloid progeny of human CD34⁺ bone marrow precursors cultured with c-kit ligand, granulocyte-macrophage colony-stimulating factor, and TNF-α.” J Immunol (1995) 154:5851-61; Caux C et al., “Tumor necrosis factor α strongly potentiates interleukin-3 and granulocyte-macrophage colony-stimulating factor-induced proliferation of human CD34⁺ hematopoietic progenitor cells.” Blood (1990) 75:2292-8; Chen B et al., “The role of tumor necrosis factor (in modulating the quantity of peripheral blood-derived, cytokine-driven human dendritic cells and its role in enhancing the quality of dendritic cell function in presenting soluble tumor antigens to CD4+ T cells in vitro.” Blood. (1998) 91(12):4652-4661. Exemplary nucleic acids encoding TNF-α and equivalents are disclosed, e.g., in Genbank accession nos. X01394, A21522, NM_{—}013693, M20155, M38296, and M11731, and in U.S. Pat. Nos. 4,677,063, 4,677,064, 4,677,197, and 5,298,407.

TRANCE has been reported to increase survival and immunostimulatory properties of dendritic cells. See, e.g., Josien F et al., “TRANCE, a tumor necrosis factor family member enhances the longevity and adjuvant properties of DCs in vivo.” J Exp Med. 2000;191(3):495-502. Exemplary nucleic acids encoding TRANCE and equivalents are disclosed, e.g., in Genbank accession nos. NM_{—}011613, AF013170, NM_{—}033012, NM_{—}003701, AF053712, AF013171, and AB037599, and in U.S. Pat. No. 6,242,586.

TRAIL has been shown to promote the ability of dendritic cells to cause apoptosis of tumor cells targets. See, e.g., Fanger N A, Maliszewski C R, Schooley K, Griffith T S. Human dendritic cells mediate cellular apoptosis via tumor necrosis factor-related apoptosis-inducing ligand (TRAIL). J Exp Med. 1999;190(8):1155-1164. Exemplary nucleic acids encoding TRAIL and equivalents are disclosed, e.g., in Genbank accession nos. U37518, NM_{—}003810 XM_{—}045049, U37522, NM_{—}009425, and AB052771, and in U.S. Pat. No. 5,763,223.

Exemplary nucleic acids encoding GM-CSF and equivalents are disclosed, e.g., in Genbank accession nos. M17706, X03655, X03438, X03656, M13926, NM_{—}009971, and X05402, and in U.S. Pat. No. 4,810,643, and in foreign patent documents WO-A-8702060, WO-A-8604605, and WO-A-8604506.

Exemplary nucleic acids encoding IL-4 and equivalents are disclosed, e.g., in Genbank accession nos. NM_{—}000589, M13982, X81851, AF395008, M23442, NM_{—}021283, M25892, X05064, X05253, and X05252, and in U.S. Pat. No. 5,017,691. See also Tarte K, Klein B. Dendritic cell-based vaccine: a promising approach for cancer immunotherapy. Leukemia. 1999;13:653-663.

c-Kit ligand has been shown to support proliferation and long-term maintenance of dendritic cells, especially in synergy with other factors. See, e.g., Szabolcs P et al., “Expansion of immunostimulatory dendritic cells among the myeloid progeny of human CD34⁺ bone marrow precursors cultured with c-kit ligand, granulocyte-macrophage colony-stimulating factor, and TNF-α.” J Immunol (1995) 154:5851-61. Exemplary nucleic acids encoding kit ligand and equivalents are disclosed, e.g., in Genbank accession nos. AF400437, AF400436, M59964, M59964, NM_{—}000899, NM_{—}003994, and U44725, and in U.S. Pat. Nos. 6,001,803 and 5,525,708.

Exemplary nucleic acids encoding IL-13 and equivalents are disclosed, e.g., in Genbank accession nos. NM_{—}002188, X69079, L06801, U10307, AF377331, NM_{—}008355, L13028, and M23504, and in U.S. Pat. Nos. 5,652,123 and 5,696,234.

Exemplary nucleic acids encoding IL-1a and equivalents are disclosed, e.g., in Genbank accession nos. NM_{—}000575, M28983, X02531, M15329, AF010237, NM_{—}013598, M57647, and X68989, and in U.S. Pat. Nos. 5,371,204, 5,008,374, 5,017,692, and 5,756,675.

Exemplary nucleic acids encoding IL-1β and equivalents are disclosed, e.g., in Genbank accession nos. X02532, M15330, and M15840, and in U.S. Pat. Nos. 5,286,847 and 5,047,505.

Exemplary nucleic acids encoding IL-6 and equivalents are disclosed, e.g., in Genbank accession nos. Y00081, X04602, M54894, M38669, and M14584, and in U.S. Pat. No. 5,338,834.

Exemplary nucleic acids encoding IL-15 and equivalents are disclosed, e.g., in Genbank accession nos. U14407, NM_{—}000585, X91233, Z38000, X94222, Y09908, U14332, NM_{—}008357, and AF038164, and in U.S. Pat. No. 5,747,024.

Exemplary nucleic acids encoding TGF-β1 and equivalents are disclosed, e.g., in Genbank accession nos. M38449, M55656, X05839, Y00112, X02812, J05114, AJ009862, M13177, and BC013738. See also, e.g., Strobl H, et al., “Flt3-ligand in cooperation with transforming growth factor-β1 potentiates in vitro development of Langherans-type dendritic cells and allows single-cell dendritic cell cluster formation under serum-free conditions.” Blood (1997) 90:1425-34; Borkowsky T A et al., “A role for endogenous transforming growth factor-β1 in Langherans cell biology: the skin of transforming growth factor-β1 null mice is devoid of epidermal Langherans cells.” J. Exp. Med. (1996) 184:4520-30.

Nucleic acids that encode molecules that block inhibitory signals are also contemplated for inclusion as Gene 2 in an expression vector. An example of an inhibitory receptor which may be blocked by an antagonist encoded as gene 2 in an exemplary expression vector is vascular endothelial growth factor receptor. See, e.g., Gabrilovich D et al., “Vascular endothelial growth factor inhibits the development of dendritic cell and dramatically affects the differentiation of multiple hematopoietic lineages in vivo.” Blood 1998; 92:4150-66.

Many of the above-mentioned ligands are known to act synergistically with one another, as described in the references cited above. Therefore, the present subject matter also contemplates expression vector embodiments comprising a tricistronic construct having a first gene expression cassette comprising an tumor antigen gene under control of an tumor antigen presenting cell-specific promoter, a second gene expression cassette comprising a factor gene that stimulates tumor antigen presenting cell differentiation, maturation, expansion or activation, and a third gene expression cassette comprising a factor gene that stimulates tumor antigen presenting cell differentiation, maturation, expansion or activation, wherein the second and third gene expression cassettes are any combination of exemplary nucleic acids or their equivalents encoding any of the exemplary molecules or their equivalents that can modulate differentiation, maturation, expansion or activation of dendritic cells or other tumor antigen presenting cells.

4.7. Vectors

The invention further provides plasmids and vectors encoding a tumor antigen or immunostimulatory protein, which can be used to express the tumor antigen or immunostimulatory protein in a host cell. The host cell may be any prokaryotic or eukaryotic cell. Thus, a nucleotide sequence derived from the cloning of mammalian Tumor antigen proteins, encoding all or a selected portion of the full-length protein, can be used to produce a recombinant form of a Tumor antigen polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Typically, expression vectors used for expressing, in vivo or in vitro an tumor antigen protein contain a nucleic acid encoding an tumor antigen polypeptide, operably linked to at least one transcriptional regulatory sequence. Regulatory sequences are art-recognized and are selected to direct expression of the subject proteins in the desired fashion (time and place). Transcriptional regulatory sequences are described in Goeddel; Gene Expression Technology: Methods in Enzymology 185, Academic Press, San Diego, Calif. (1990).

Suitable vectors for the expression of a tumor antigen polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In a preferred embodiment, the promoter is a constitutive promoter, e.g., a strong viral promoter, e.g., CMV promoter. The promoter can also be cell- or tissue-specific, that permits substantial transcription of the DNA only in predetermined cells, e.g., in professional tumor antigen presenting cells, such as a promoter specific for fibroblasts, or smooth muscle cells, retinal cells or RPE cells. A smooth muscle specific promoter is, e.g., the promoter of the smooth muscle cell marker SM22alpha (Akyura et al., (2000) Mol Med 6:983. Retinal pigment epithelial cell specific promoter is, e.g., the promoter of the Rpe65 gene (Boulanger et al. (2000) J Biol Chem 275:31274). The promoter can also be an inducible promoter, e.g., a metallothionein promoter. Other inducible promoters include those that are controlled by the inducible binding, or activation, of a transcription factor, e.g., as described in U.S. Pat. Nos. 5,869,337 and 5,830,462 by Crabtree et al., describing small molecule inducible gene expression (a genetic switch); International patent applications PCT/US94/01617, PCT/US95/10591, PCT/US96/09948 and the like, as well as in other heterologous transcription systems such as those involving tetracyclin-based regulation reported by Bujard et al., generally referred to as an allosteric “off-switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. Other inducible transcription systems involve steroid or other hormone-based regulation.

The polynucleotide of the invention together with all necessary transcriptional and translational control sequences is referred to herein as “construct of the invention” or “transgene of the invention.”

The polynucleotide of the invention may also be introduced into the cell in which it is to be expressed together with another DNA sequence (which may be on the same or a different DNA molecule as the polynucleotide of the invention) coding for another agent. Exemplary agents are further described below. In one embodiment, the DNA encodes a polymerase for transcribing the DNA, and may comprise recognition sites for the polymerase and the injectable preparation may include an initial quantity of the polymerase.

In certain instances, it may be preferred that the polynucleotide is translated for a limited period of time so that the polypeptide delivery is transitory. This can be achieved, e.g., by the use of an inducible promoter.

The polynucleotides used in the present invention may also be produced in part or in total by chemical synthesis, e.g., by the phosphoramidite method described by Beaucage and Carruthers, Tetra. Letts., 22:1859-1862 (1981) or the triester method according to the method described by Matteucci et al., J. Am. Chem. Soc., 103:3185 (1981), and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

The polynucleotide of the invention operably linked to all necessary transcriptional and translational regulation elements can be injected as naked DNA into a subject. In a preferred embodiment, the polynucleotide of the invention and necessary regulatory elements are present in a plasmid or vector. Thus, the polynucleotide of the invention may be DNA, which is itself non-replicating, but is inserted into a plasmid, which may further comprise a replicator. The DNA may be a sequence engineered so as not to integrate into the host cell genome.

Preferred vectors for use according to the invention are expression vectors, i.e., vectors that allow expression of a nucleic acid in a cell vectors. Preferred expression vectors are those which contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and phyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al. Colloidal dispersion systems.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below). For example, smooth muscle cells can be targeted with an antibody binding specifically to SM22α, a smooth muscle cell marker. Retinal cells and RPE cells can similarly be targeted.

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans. The following additional guidance on the choice and use of viral vectors may be helpful to the practitioner. As described in greater detail below, such embodiments of the subject expression constructs are specifically contemplated for use in various in vivo and ex vivo gene therapy protocols.

4.8 Pharmaceutical Compositions and Formulations

The invention provides pharmaceutical compositions comprising the above-described vaccine and tropic immunostimulatory agents. In one aspect, the present invention provides pharmaceutically acceptable compositions which comprise a therapeutically-effective amount of one or more of the compounds described above, formulated together with one or more pharmaceutically acceptable carriers (additives) and/or diluents. In another aspect, certain embodiments, the compounds of the invention can be administered as such or in admixtures with pharmaceutically acceptable carriers and can also be administered in conjunction with other chemotherapeutic agents. Conjunctive (combination) therapy thus includes sequential, simultaneous and separate, or co-administration of the active compound in a way that the therapeutic effects of the first administered one is not entirely disappeared when the subsequent is administered.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) tropical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

In one embodiment, the pharmaceutical compositions are formulated for parenteral administration. In one embodiment, the pharmaceutical composition is formulated for intraarterial injection. In another preferred embodiment, the pharmaceutical compositions are formulated for systemic administration.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, tropical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the tropical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, tropically, or rectally. They are of course given in forms suitable for each administration route. For example, they are administered in tablets or capsule form, by injection, inhalation, eye lotion, ointment, suppository, etc. administration by injection, infusion or inhalation; tropical by lotion or ointment; and rectal by suppositories. Oral administrations are preferred.

These compounds may be administered to humans and other animals for therapy by any suitable route of administration, including orally, nasally, as by, for example, a spray, rectally, intravaginally, parenterally, intracistemally and tropically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

While it is possible for a compound of the present invention to be administered alone, it is preferable to administer the compound as a pharmaceutical formulation (composition). The compounds according to the invention may be formulated for administration in any convenient way for use in human or veterinary medicine, by analogy with other pharmaceuticals.

In certain embodiments, the above-described pharmaceutical compositions comprise one or more of the inhibitors, a second chemotherapeutic agent, and optionally a pharmaceutically acceptable carrier.

The term traditional chemotherapeutic agents include, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following additional drugs may also be used in combination with these antineoplastic agents, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons a, b, and g; hormones, such as luteinizing hormone releasing hormone (LHRH) and analogues and, gonadotropin releasing hormone (GnRH); growth factors, such as transforming growth factor-b (TGF-b), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-a & b (TNF-a & b); invasion inhibiting factor-2 (IIF-2); bone morphogenetic proteins 1-7 (BMP 1-7); somatostatin; thymosin-a-1; g-globulin; superoxide dismutase (SOD); complement factors; anti-angiogenesis factors; tumor antigenic materials; and pro-drugs.

In a preferred embodiment, the composition of the invention may comprise other biologically active substances, preferably a therapeutic drug or pro-drug, for example, other chemotherapeutic agents, scavenger compounds, antibiotics, anti-virals, anti-fungals, anti-inflammatories, vasoconstrictors and anticoagulants, tumor antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically activated when implanted, injected or otherwise inserted into the tumor.

4.9 Therapeutic Methods

The present invention further provides novel therapeutic methods of treating a cancerous tumor comprising administering to the subject an effective amount of a subject pharmaceutical composition. The methods of the present invention may be used to treat any cancerous tumor. In certain embodiments, the method comprises parenterally administering an effective amount of a subject pharmaceutical composition to a subject. In one embodiment, the method comprises intraarterial administration of a subject composition to a subject. In other embodiments, the method comprises administering an effective amount of a subject composition directly to the arterial blood supply of a cancerous tumor in a subject. In one embodiment, the methods comprises administering an effective amount of a subject composition directly to the arterial blood supply of the cancerous tumor using a catheter. In embodiments where a catheter is used to administer a subject composition, the insertion of the catheter may be guided or observed by fluoroscopy or other method known in the art by which catheter insertion may be observed and/or guided. In another embodiment, the method comprises chemoembolization. For example a chemoembolization method may comprise blocking a vessel feeding the cancerous tumor with a composition comprised of a resin-like material mixed with an oil base (e.g., polyvinyl alcohol in Ethiodol) and one or more chemotherapeutic agents. In still other embodiments, the method comprises systemic administration of a subject composition to a subject.

In certain embodiments, the methods of treating a cancerous tumor comprise administering one or more selective inhibitors of the invention in conjunction with a second agent to a subject. Such methods in certain embodiments comprise administering pharmaceutical compositions comprising one or more inhibitors in conjunction with other chemotherapeutic agents or scavenger compounds. Conjunctive therapy includes sequential, simultaneous and separate, or co-administration of the active compound in a way that the therapeutic effects of the first administered one is not entirely disappeared when the subsequent is administered. In one embodiment, the second agent is a chemotherapeutic agent. In another embodiment, the second agent is a scavenger compound. In certain embodiments, the second agent may be formulated into a separate pharmaceutical composition. In other embodiments, the pharmaceutical composition may comprise both an inhibitor and a second agent.

In other embodiments, the methods of treating a cancerous tumor comprise administering an effective amount of a subject composition directly to the blood vessels in the liver, head, neck, glands, or bones. For example, blood vessels such as the hepatic, femoral, cerebral, carotid, or vertebral arteries may be infused, injected, chemoembolized, or catheterized to administer the subject compositions to a cancerous tumor. In other embodiments, the methods comprise administering an effective amount of a subject composition directly to the blood vessels in a cancerous tumor in the head, neck, or bones. Such methods are well-known and used in the art. For example, Gobin, Y. P, et al (2001) Radiology 218:724-732 teaches a method for interarterial chemotherapy for brain tumors. Moser, et al. (2002) Head Neck 24:566-74 reviews the use of intraarterial catheters for chemotherapeutic treatment in head and neck cancer. Wang, M. Q., et al. (2001) J. Vasc. Interv. Radiol. 12:731-7 teaches a method of injecting the femoral arteries as well as a method of chemoembolization in order to treat osteosarcoma. Kato, T., et al. (1996) Cancer Chemother Pharmacol 37(4):289-96 reviews the use of intraarterial infusion of microencapsulated anticancer drugs (chemoembolization) to treat cancerous tumors in the liver, kidney, intrapelvic organs, lung, head and neck, and bones. Hermann, K., et al (2000) Radiology 215:294-9; Kemeny, N. E., (1999) Baillieres Best Pract Res Clin Gastroenterol 13:593-610 describe exemplary methods of intraarterial and embolization methods for treatment of liver cancer.

In general, chemoembolization or direct intraarterial or intravenous injection therapy utilizing pharmaceutical compositions of the present invention is typically performed in a similar manner, regardless of the site. Briefly, angiography (a road map of the blood vessels), or more specifically in certain embodiments, arteriography, of the area to be embolized may be first performed by injecting radiopaque contrast through a catheter inserted into an artery or vein (depending on the site to be embolized or injected) as an X-ray is taken. The catheter may be inserted either percutaneously or by surgery. The blood vessel may be then embolized by refluxing pharmaceutical compositions of the present invention through the catheter, until flow is observed to cease. Occlusion may be confirmed by repeating the angiogram. In embodiments where direct injection is used, the blood vessel is then infused with a pharmaceutical composition of the invention in the desired dose.

Embolization therapy generally results in the distribution of compositions containing inhibitors throughout the interstices of the tumor or vascular mass to be treated. The physical bulk of the embolic particles clogging the arterial lumen results in the occlusion of the blood supply. In addition to this effect, the presence of an anti-angiogenic factor(s) prevents the formation of new blood vessels to supply the tumor or vascular mass, enhancing the devitalizing effect of cutting off the blood supply. Direct intrarterial or intravenous generally results in distribution of compositions containing inhibitors throughout the interstices of the tumor or vascular mass to be treated as well. However, the blood supply is not generally expected to become occluded with this method.

Within one aspect of the present invention, primary and secondary tumors of the liver or other tissues may be treated utilizing embolization or direct intraarterial or intravenous injection therapy. Briefly, a catheter is inserted via the femoral or brachial artery and advanced into the hepatic artery by steering it through the arterial system under fluoroscopic guidance. The catheter is advanced into the hepatic arterial tree as far as necessary to allow complete blockage of the blood vessels supplying the tumor(s), while sparing as many of the arterial branches supplying normal structures as possible. Ideally this will be a segmental branch of the hepatic artery, but it could be that the entire hepatic artery distal to the origin of the gastroduodenal artery, or even multiple separate arteries, will need to be blocked depending on the extent of tumor and its individual blood supply. Once the desired catheter position is achieved, the artery is embolized by injecting compositions (as described above) through the arterial catheter until flow in the artery to be blocked ceases, preferably even after observation for 5 minutes. Occlusion of the artery may be confirmed by injecting radio-opaque contrast through the catheter and demonstrating by fluoroscopy or X-ray film that the vessel which previously filled with contrast no longer does so. In embodiments where direct injection is used, the artery is infused by injecting compositions (as described above) through the arterial catheter in a desired dose. The same procedure may be repeated with each feeding artery to be occluded.

For use in embolization therapy, compositions of the present invention are preferably non-toxic, thrombogenic, easy to inject down vascular catheters, radio-opaque, rapid and permanent in effect, sterile, and readily available in different shapes or sizes at the time of the procedure. In addition, the compositions preferably result in the slow (ideally, over a period of several weeks to months) release of an inhibitor and/or a second agent. Particularly preferred compositions should have a predictable size of 15-200 microns after being injected into the vascular system. Preferably, they should not clump into larger particles either in solution or once injected. In addition, preferable compositions should not change shape or physical properties.

In most embodiments, the subject pharmaceutical compositions will incorporate the substance or substances to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

For the subject compositions, a range of dosage is contemplated by the present invention. The present invention contemplates embodiments that release at least those amounts over a three week period, at least twice those amounts over a six week period, etc.

Dosage may be based on the amount of the composition per kg body weight of the patient. For example, a range of amounts of compositions are contemplated, including about 0.001, 0.01, 0.1, 0.5, 1, 10, 15, 20, 25, 50 mg or more of such compositions per kg body weight of the patient. Other amounts will be known to those of skill in the art and readily determined.

In certain embodiments, the dosage of the subject compounds will generally be in the range of about 0.001 mg to about 10 mg per kg body weight, specifically in the range of about 0.1 mg to about 10 mg per kg, and more specifically in the range of about 0.1 mg to about 1 mg per kg. In one embodiment, the dosage is in the range of about 0.3 mg to about 0.6 mg per kg. In one embodiment, the dosage is in the range of about 0.4 mg to about 0.5 mg per kg.

Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED50 with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

4.10 Kits

The present invention provides kits for treating various cancers. For example, a kit may comprise one or more pharmaceutical compositions as described above. The compositions may be pharmaceutical compositions comprising a pharmaceutically acceptable excipient. In other embodiments involving kits, this invention provides a kit including pharmaceutical compositions of the present invention, and optionally instructions for their use. In still other embodiments, the invention provides a kit comprising one or more pharmaceutical compositions and one or more devices for accomplishing administration of such compositions. For example, a subject kit may comprise a pharmaceutical composition and catheter for accomplishing direct intraarterial injection of the composition into a cancerous tumor. In one embodiment, the device is an intraarterial catheter. Such kits may have a variety of uses, including, for example, therapy, diagnosis, and other applications.

### 5. EXAMPLE

The examples below provide guidance to the skilled artisan in applying the methods and compositions of the invention for treating cancer, including neoplasms and metastatic tumors, using a combination of a GM-CSF secreting tumor cell vaccine and an attenuated tropic bacteria that localizes or can be localized to the affected cancerous site. In particular, the examples below demonstrate that treatment of a hepatic metastatic cancer with a combination of a GM-CSF secreting tumor cell vaccine is augmented by the delivery of an attenuated strain of bacteria that localizes to the liver, but does not augment the action of the GM-CSF secreting tumor cell vaccine to other tissues to which the attenuated strain of bacteria does not localize. Accordingly, the examples below provide broad support for a combination method of treating disease, particularly cancers, that includes a systemic cancer vaccine and an agent that is tropic to the disease-affected organ or tissues (e.g. a tropic attenuated bacteria or virus or other such agent that can be localized by direct application to the disease-affected region).

5.1 Murine Hepatic Metastasis Model

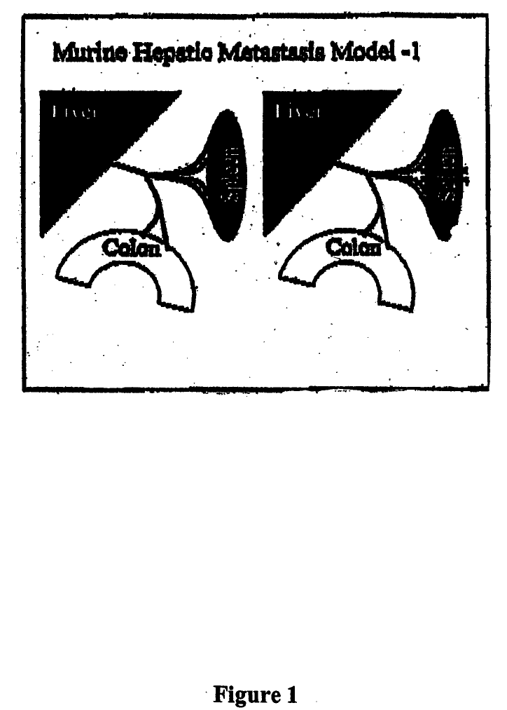

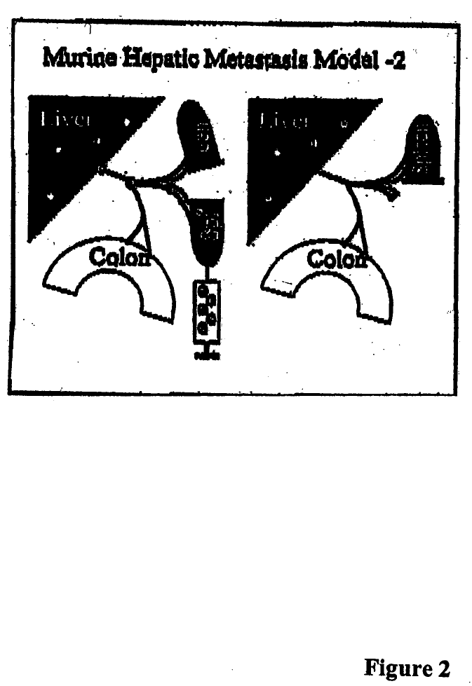

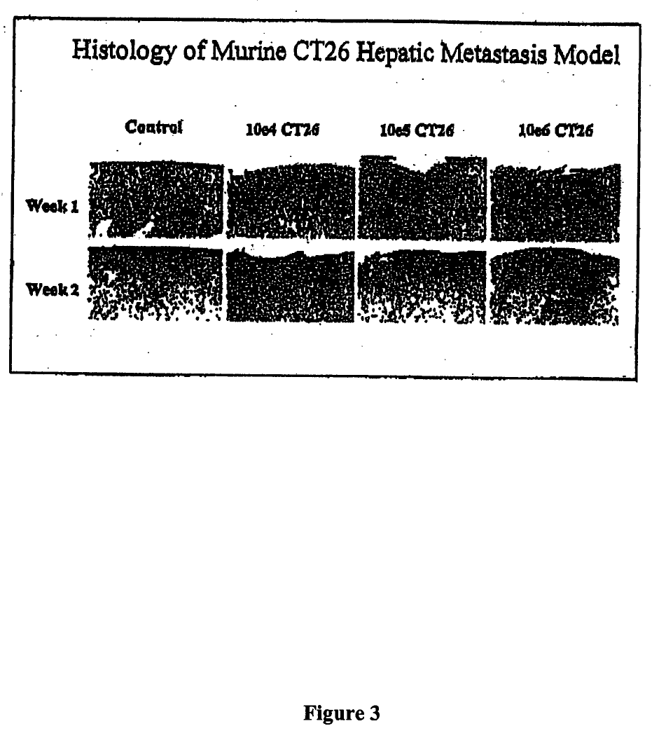

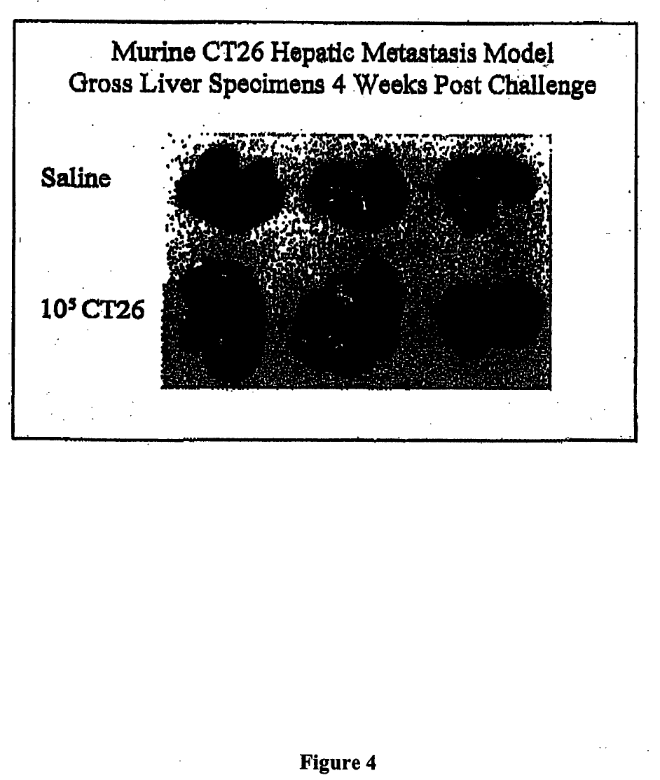

Six to eight week old female BALB/c mice were purchased from the National Cancer Institute and were used for the following experiments with CT26. CT26 is a murine colorectal cancer tumor cell line derived from BALB/c mice. The mice were anesthetized with pentobarbital (50 mg/kg intraperitoneal). For each mouse, laparotomy was performed to expose the spleen. The spleen was divided into two hemi-spleens using titanium clips, leaving the vascular pedicles intact. (see FIG. 1). A 27 gauge needs was used inject 0, 1×10⁴, 1×10⁵, 1×10⁶ CT26 cells in 300 μl of Hanks Balanced Salt Solution (HBSS) into one of the hemi-spleens. Cells then flowed into the splenic and portal veins, and formed tumor deposits in the liver. The CT26-contaminated hemi-spleen was than surgically removed, leaving a functional hemi-spleen free of tumor cells. (see FIG. 2). The mice were then closed and recovered from anesthesia. At one and two weeks, three mice from each group of tumor challenge were euthanized using CO2 inhalation. The livers were removed from the mice. Additionally, the livers were sectioned and H+E stained to determine the absence or presence of microscopic tumor burden. (see FIG. 3) These analyses revealed no microscopic or visible tumor nodules in mice receiving no tumor or 1×10⁴ CT26 cells. When 1×10⁵ CT26 tumor cells were injected, small microscopic foci of tumor were seen under the microscope at one week and became larger at two weeks. These foci were not visible to the eye at one week and could be seen at two weeks. When 1×10⁶ CT26 tumor cells were injected, tumor nodules were easily seen on microscopy. FIG. 4 demonstrates two groups of livers from mice that were euthanized four weeks after challenge with saline or 1×10⁵ CT26 in the hepatic metastasis model described above. The whitish cancer nodules are apparent in the CT26 challenged mice.

5.2 Protective Effect of a GM-CSF Secreting Tumor Cell Vaccine

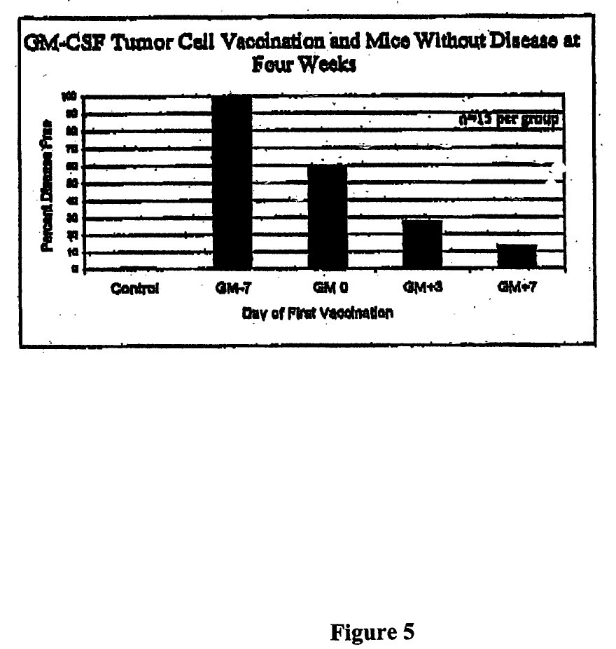

In this set of experiments, mice were challenged with 1×10⁵ CT26 cells via the spleen as outlined in the hepatic metastasis model. Mice were either vaccinated with Hanks balanced Salt Solution (HBSS) or vaccinated with 1×10⁶ irradiated (5000 rad) GM-CSF secreting gene modified CT26 (GM/CT26) cells biweekly beginning seven days before CT26 tumor challenge, on the day of tumor challenge, three days after tumor challenge or seven days after tumor challenge. At four weeks, mice were euthanized and their livers analyzed for the presence of metastatic disease both grossly and microscopically. FIG. 5 summarizes the data from two experiments.

Control mice that did not receive GM/CT26 vaccination all developed hepatic metastases. None of the mice that received the vaccination beginning seven days before tumor challenge developed hepatic metastases. Nine of fifteen mice that received vaccination on the day of tumor challenge were free of hepatic metastases. Four of fifteen mice that received vaccination beginning three days after tumor challenge were free of hepatic metastases. Two of fifteen mice that received vaccination beginning seven days after tumor challenge were free of hepatic metastases.

5.3 Augmentation of GM-CSF Tumor Vaccine Efficacy by Attenuated Listeria

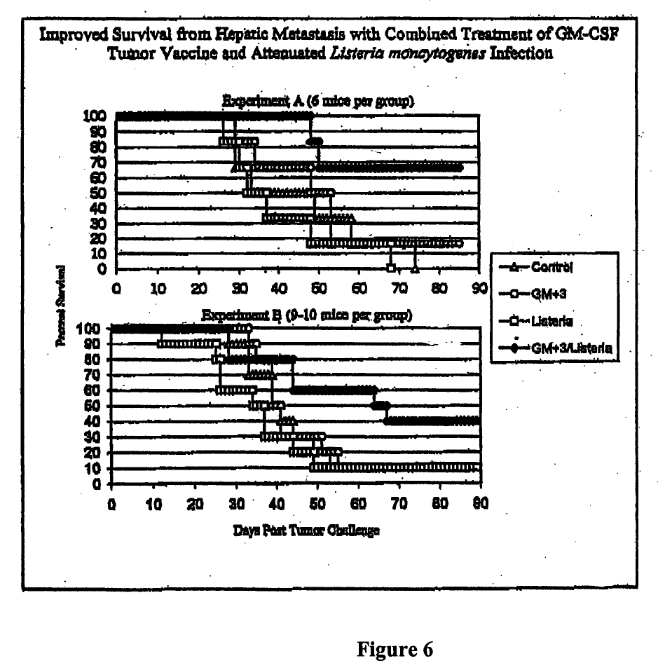

Experiments were conducted to test the efficacy of GM-CSF secreting tumor cell vaccines in combination with the HIV-gag attenuated strain of Listeria monocytogenes (LM). In Experiment A of FIG. 6, mice were challenged with hepatic metastases as previously described. Twenty-four mice were divided into four groups of six mice and given the following treatments:
Control: No Treatment
   GM +3: Biweekly 1×10⁶ irradiated (5000 rad) GM/CT26 for 3 weeks beginning 3 days after tumor challenge
Listeria: An intraperitoneal inoculation of 1×10⁶ colony forming units (CFU) of LM beginning 6 days after tumor challenge
GM +3/Listeria: Combination therapy of GM/CT26 vaccine and LM inoculation as described above.

Three of the Control and Listeria mice were dead by day 33, and all of them were dead by days 74 and 68, respectively. The GM +3 mice had slightly improved survivals with three of six dead at day 48 and one mouse surviving long-term. The GM+3/Listeria Group had markedly improved survival with four of the six surviving long-term.

The results of this study were repeated with nine or ten mice per group. These results are summarized in Experiment B of FIG. 6. Five of ten of the Control, Listeria and GM +3/Listeria mice were dead at day 64. This group also had four of the ten surviving long-term whereas the other three groups only had one mouse surviving long-term. The results indicate that LM treatment increases the efficacy of GM-CSF secreting tumor cell vaccines initiated against established hepatic metastases.

5.4 Augmentation of GM-CSF Tumor Vaccine Efficacy by Attenuated Listeria monocytogenes Infection is Specific to the Liver and Not the Lung

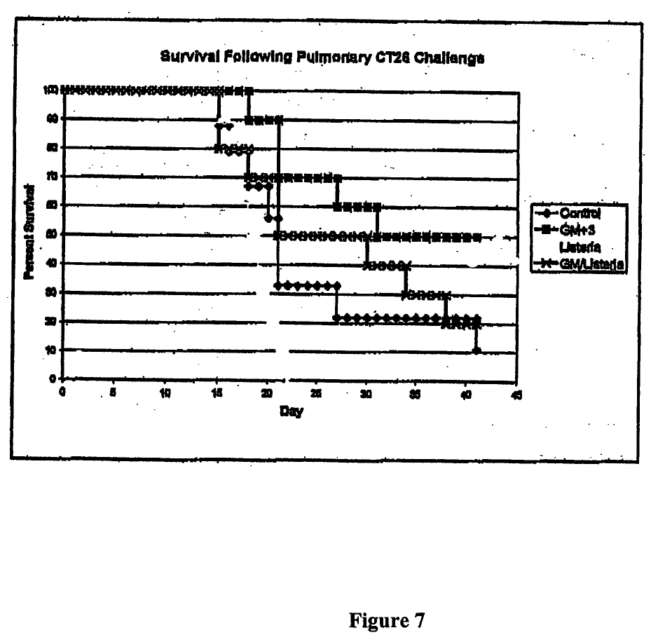

Experiments were conducted to test the efficacy of GM-CSF secreting tumor cell vaccines in combination with HIV-gag attenuated strain of Listeria monocytogenes (LM) in a pulmonary colorectal metastasis model. In this experiment (FIG. 7), mice were challenged with pulmonary metastases by giving a tail vein injection of 5×10⁵ CT26 cells. Each of the following groups had nine or ten mice each:
Control: No treatment
GM +3: Biweekly 1×10⁶ irradiated (5000 rad) GM/CT26 for 3 weeks beginning 3 days after tumor challenge
Listeria: An intraperitoneal inoculation of 1×106 colony forming units (CFU) of LM beginning 6 days after tumor challenge
GM +3/Listeria: Combination therapy of GM/CT26 vaccine and LM inoculation as described above.

The GM+3/Listeria mice did not fare any better than the GM+3 alone treated mice. The survival curves of the GM+3/Listeria group and the Control group were quite similar and inferior to the survival curve of the GM+3 alone group.

5.5 Further Listeria Augmentation and Specificity Studies

We repeated the experiments demonstrating augmentation of the liver tumor vaccination by injection of Listeria. FIG. 9 shows a comparison of the survival of hepatic tumor bearing mice treated with either vaccine, Listeria, or a combination of tumor vaccine and Listeria. Mice were given hepatic tumor challenge of 1×10⁵ CT26 cells on day 0. Mice were treated with either twice weekly vaccinations initiated on day 3 for a total of 3 weeks, a single dose of 1×10⁶ Listeriae given on day 6, or combination vaccination and Listeria infection and their survival was followed.

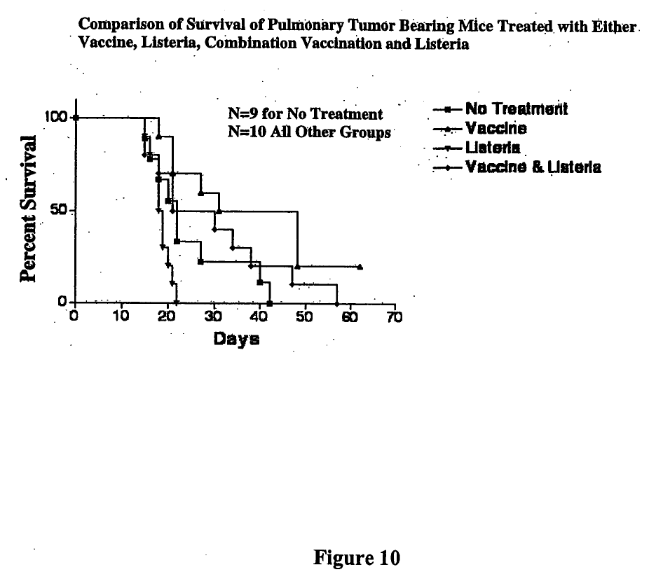

We further confirmed the specificity of the Listeria augmentation affect by examining the effect of Listeria injection on survival in a pulmonary CT26-induced tumor model. FIG. 10 shows a comparison of survival of pulmonary tumor bearing mice treated with either vaccine, Listeria, or a combination of vaccination and Listeria. Mice were given a pulmonary tumor challenge of 1×10⁵ CT26 cells on day 0. Mice were then treated with either twice weekly vaccinations initiated on day 3 for a total of 3 weeks, a single dose of 1×10⁶ Listeriae given on day 6, or a combination of vaccination and Listeria infection. Survival was then followed. Because most bacteria that enter the bloodstream are taken up and eliminated within the liver (see e.g. Gregory et al. (2002) J Leukoc Biol 72: 239-48 for review), the peritoneal injection of Listeria bacteria did not augment survival in the pulmonary tumor model.

5.6 Analysis of AH1 Tumor Antigen-Specific CD8 T-Cell Infiltration of Liver

In order to further analyze the mechanism of action of the Listeria- augmented immune response of the tumor vaccine, we first compared the levels of liver infiltrating AH1-specific CD8 T cells in the various treatment groups. FIG. 8 shows a comparison of liver infiltrating CD8 T-cells specific for AH1 tumor antigen in the various treatment groups. Mice were divided into 3 treatment groups. All mice were sacrificed on day 14. Group 1 received tumor challenge only on day 0. Group 2 received no tumor challenge, and vaccination on days 3, 7 and 11. Group 3 received tumor challenge on day 0 followed by vaccinations of days 3, 7, and 11. After sacrifice on day 14, mouse livers were digested using collagenase and hyaluronidase and centrifuged on a Ficoll density gradient in order to isolate lymphocytes. CD8 lymphocytes were analyzed for tumor antigen specificity by staining with an LdIg dimer loaded with either AH1 tumor antigen peptide or control peptide B gal.

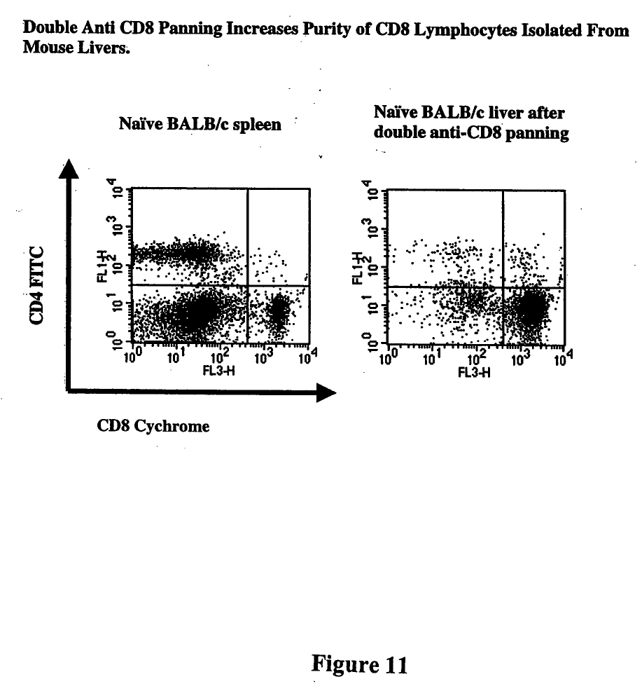

In order to still further analyze the mechanism of action of the Listeria—augmented immune response of the tumor vaccine, we performed double anti CD8 panning in order to increase the purity of CD8 lymphocytes isolated from the mouse livers in the hepatic tumor model (see FIG. 11). Pure CD8 T-cell isolates were essential in order to visualize the tumor antigen specific CD8 populations using AH1 loaded tetramers, since excess liver debris caused high background staining. Initially, livers were processed using 50 micron Medicon filters, followed by serial filtration through 100 micron and 70 micron syringe filters. Each processed liver was then plated onto a 2.43 anti-CD8 antibody coated flask for 40 minutes. The supernate was aspirated, and the flask was washed once with FACS buffer. Adherent cells were then removed using a cell scraper into 5 ml of FACS buffer, and transferred to a second antibody coated flask. After the second panning, cells were analyzed by FACS analysis. Later, a more efficient isolation process was developed. Livers were processed by straining them through a 100 micron screen filter, and then centrifuging on a 33% Percoll gradient. Lymphocytes were precipitated in the pellet. The purity of the lymphocyte pellets after Percoll centrifugation was such that only one panning was adequate to remove any excess liver debris.

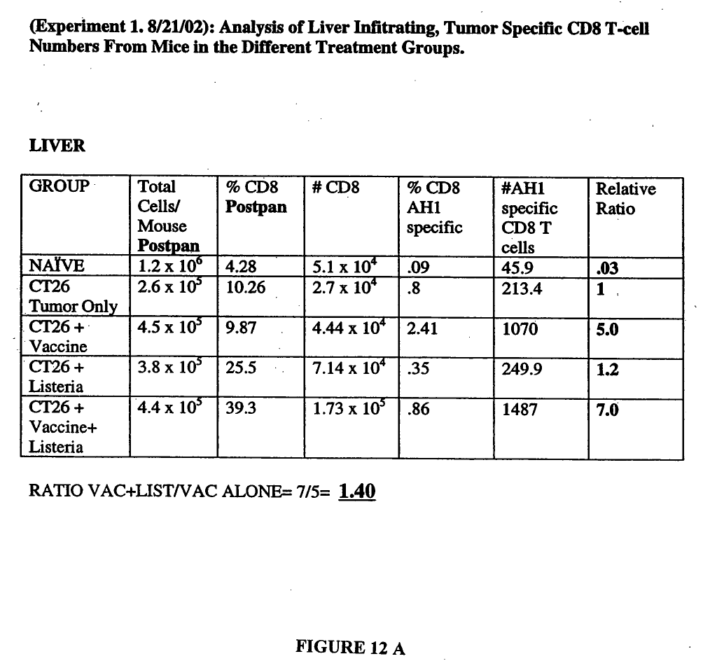

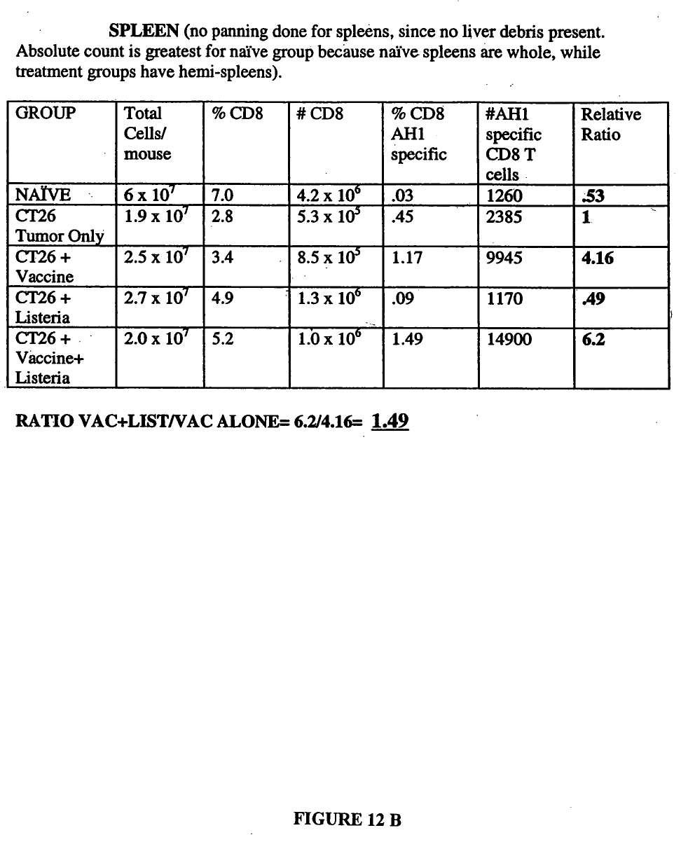

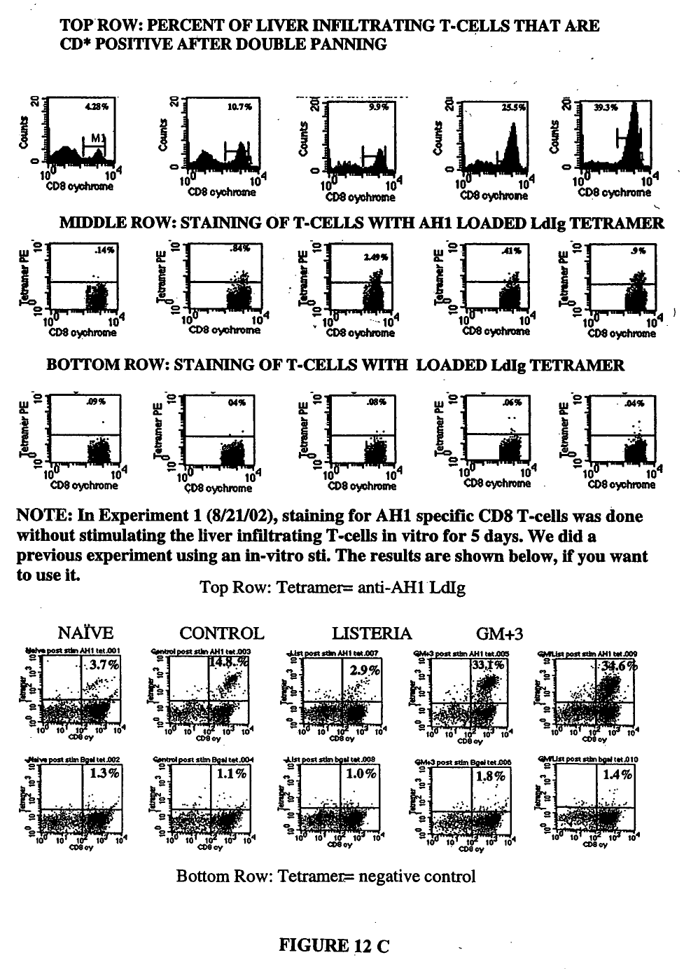

FIG. 12 shows a first experiment in which an analysis of liver infiltrating, AH1 tumor antigen-specific CD8 T-cell numbers from mice in the different treatment groups was made. Mice were given hepatic tumor challenge on day 0. Mice were treated with either vaccination alone initiated on day+3, Listeria alone on day+6, or combination vaccination and Listeria. All mice treated with vaccines received a booster vaccine on day+6. Mice were sacrificed on day 14, and liver infiltrating T-cells were isolated by using a Medicon processor and double anti-CD8 panning. FIG. 12(A) shows—analysis of the liver infiltrating lymphocytes as follows: Column 2 of the table shows the absolute number of lymphocytes per liver of mice in the different treatment groups. These counts were done after double anti-CD8 panning. Column 3 of the table shows the percentage of the lymphocytes that were CD8+. This percentage does not represent the absolute percentage in-vivo, since FACS staining to determine relative percentages was done after panning. Column 4 shows the calculated number of CD8 T-cells per liver. Column 5 shows the percentage of CD8 T-cells that were AH1 tumor antigen specific. This percentage does not reflect the actual percentage in-vivo, since FACS staining was done after panning. Column 6 shows the calculated number of AH1 specific T-cells per liver, and column 7 shows the ratio of AH1 specific cells relative to the number in untreated control mice. FIG. 12(B) shows the analysis of splenic lymphocytes. FIG. 12(C) shows FACS staining of the anti-CD8 panned, liver infiltrating T-cells isolated from mice in the different treatment groups.

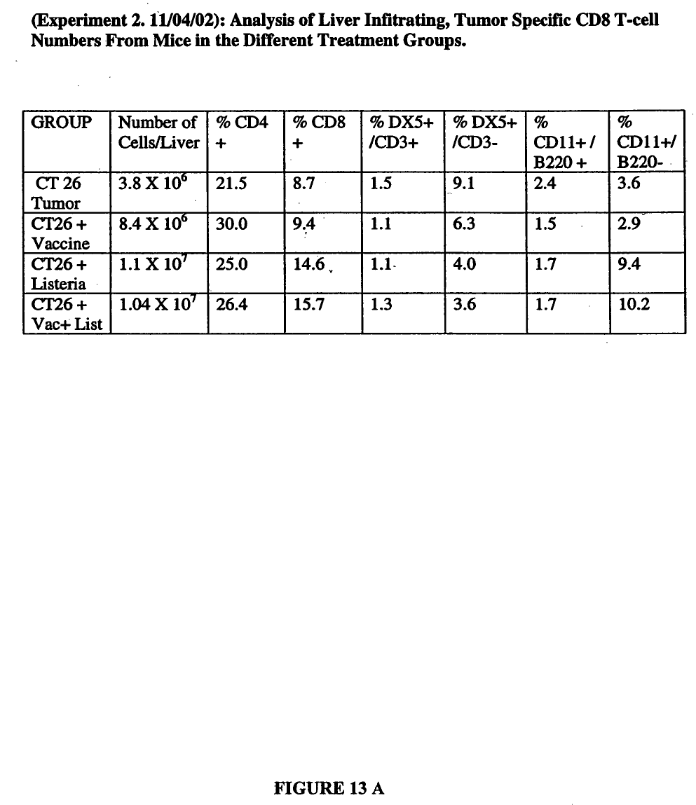

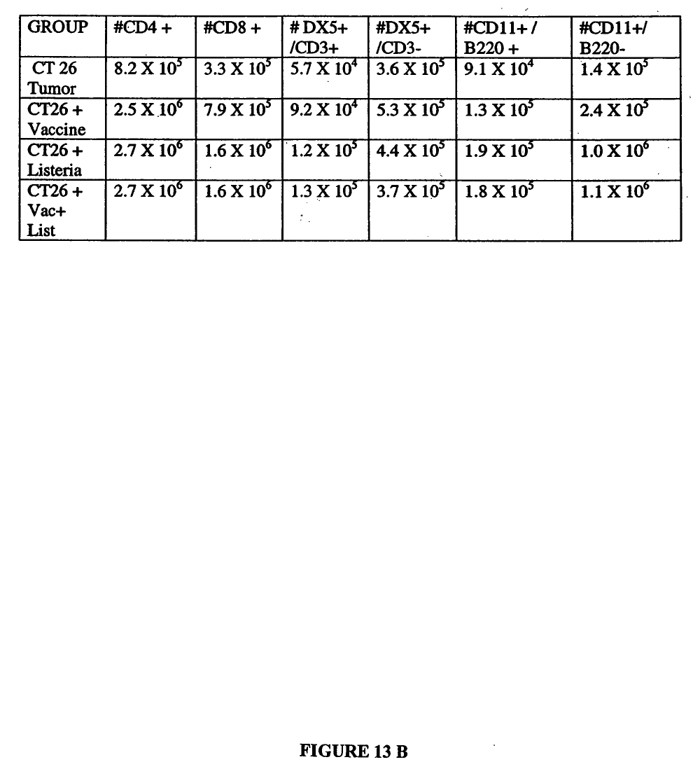

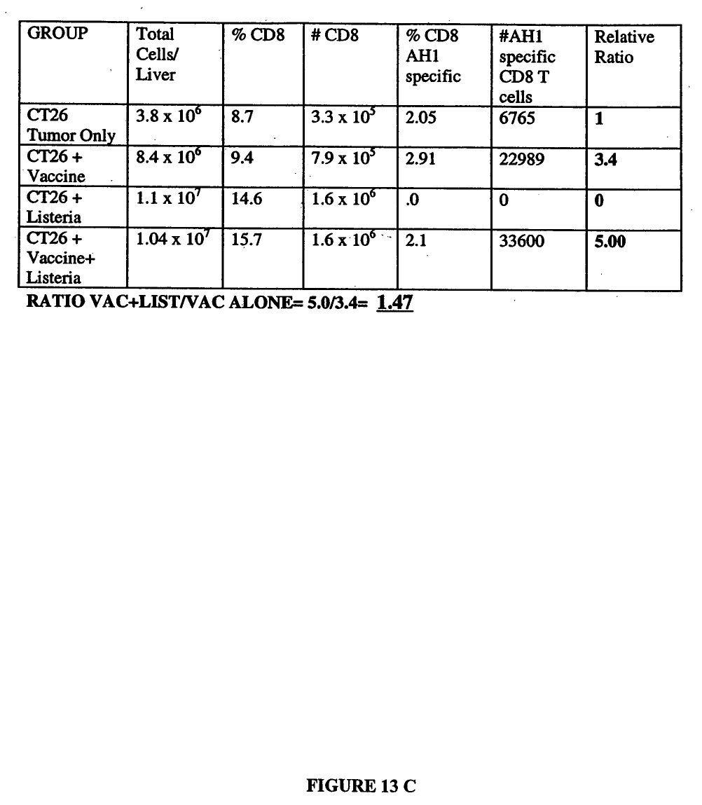

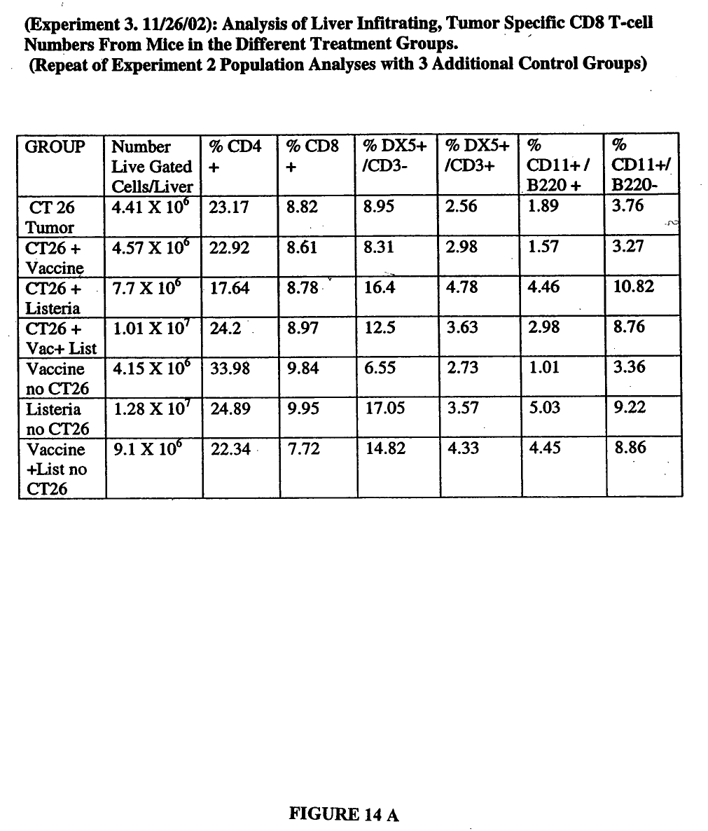

FIG. 13 shows a second experiment in which an analysis of liver infiltrating, tumor-specific CD8 T-cell numbers from mice in the different treatment groups was made. Mice were given hepatic tumor challenge on day 0. Mice were treated with either vaccination alone initiated on day+3, Listeria alone on day+6, or a combination of the vaccination and Listeria. All mice treated with vaccines received a booster vaccine on day+6. Mice were sacrificed on day 14, and liver infiltrating T-cells were isolated by straining through a 100 micron screen and filtering on a Percoll density gradient. This technique yielded much purer cellular isolates prior to panning. Liver infiltrating cell populations could be studied b FACS staining prior to panning. FIG. 13(A) is a table showing the absolute number of liver infiltrating cells per liver prior to panning, as well as the percentages of different populations in-vivo. FIG. 13(B) is a table showing the calculated absolute numbers of different cell types per liver of mice in each treatment group. In FIG. 13(C), column 2 of the table shows the absolute number of lymphocytes per liver of mice in the different treatment groups. These counts were done prior to double anti-CD8 panning. Column 3 of the table shows the percentage of the lymphocytes that were CD8+. Unlike experiment 1 (Aug. 21, 2002) this percentage does represent the absolute percentage in-vivo, since FACS staining to determine relative percentages was performed before panning. Column 4 shows the calculated number of CD8 T-cells per liver. Column 5 shows the percentage of CD8 T-cells that were AH1tumor antigen specific. Column 6 shows the calculated number of AH1 specific T-cells per liver, and column 7 shows the ratio of AH1 specific cells relative to the number in untreated control mice. The ratios calculated are very similar to the ratios from the first experiment.

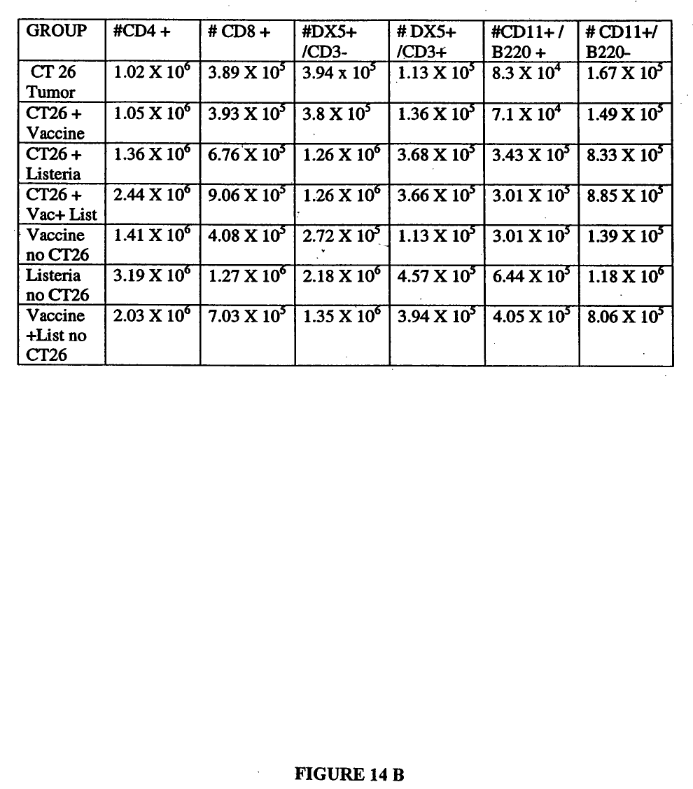

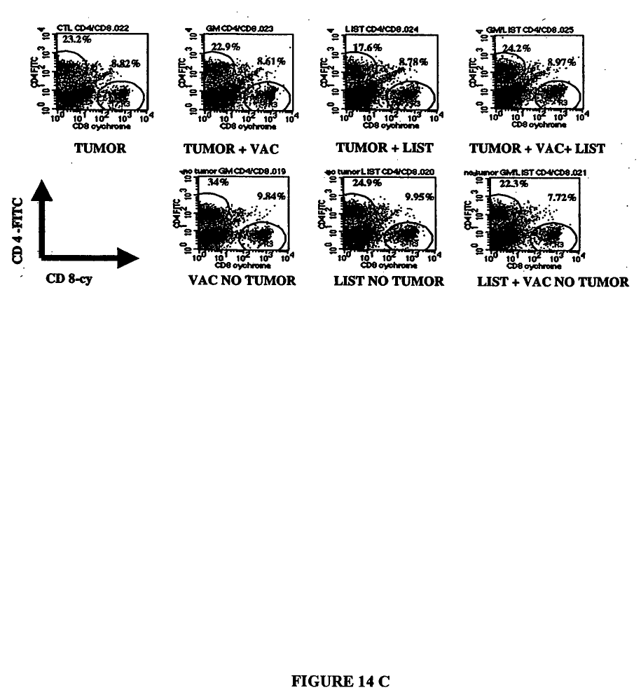

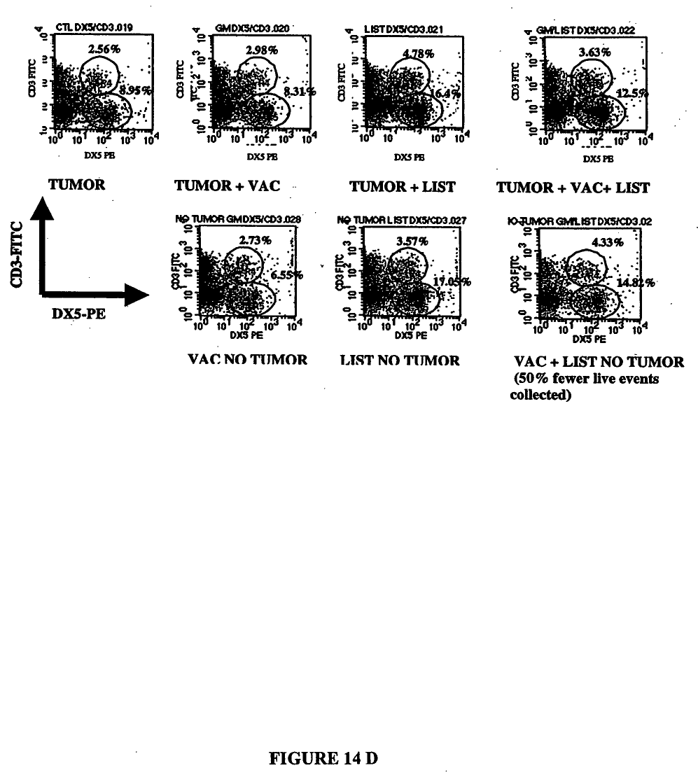

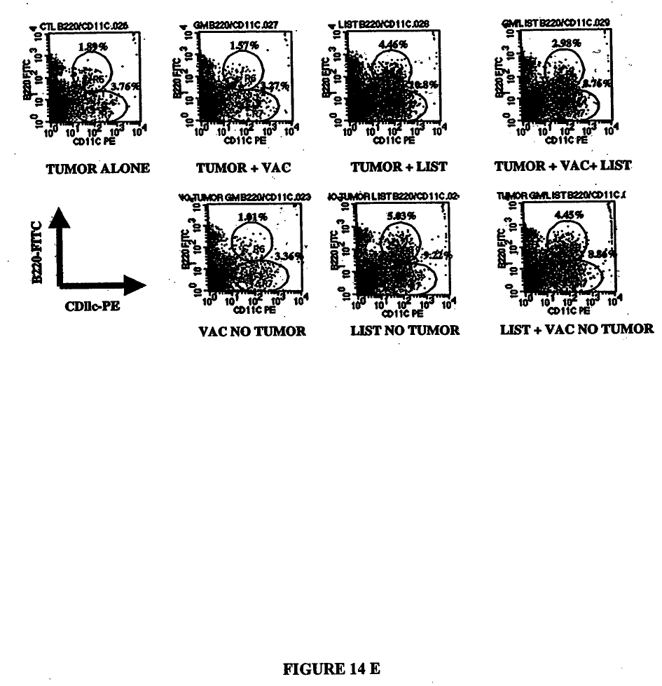

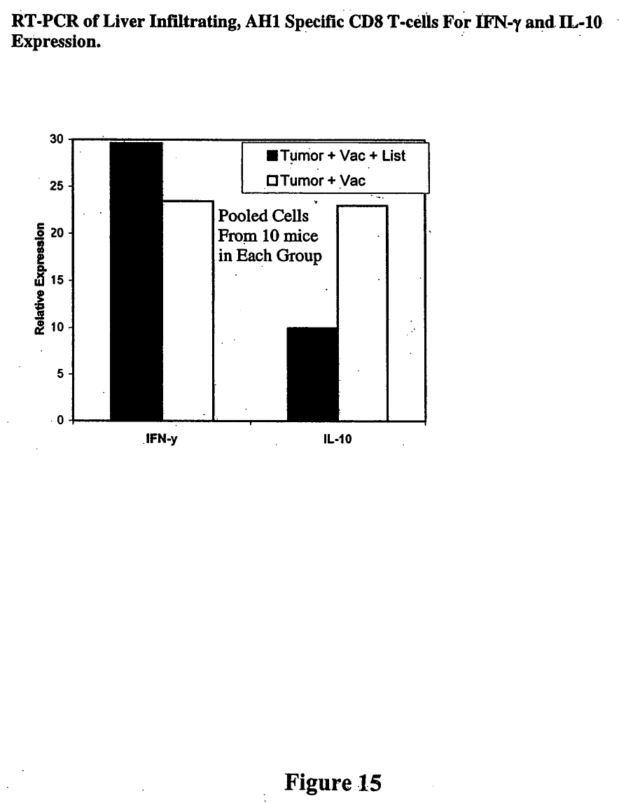

FIG. 14 shows a third experiment in which an analysis of liver infiltrating, tumor-specific CD8 T-cell numbers from mice in the different treatment groups was made. Mice were given hepatic tumor challenge on day 0. Mice were treated with either vaccination alone initiated on day+3, Listeria alone on day+6, or combination vaccination and Listeria. All mice treated with vaccines received a booster vaccine on day+6. Mice were sacrificed on day 14, and liver infiltrating T-cells were isolated by straining through a 100 micron screen and filtering on a Percoll density gradient. This technique yielded much purer cellular isolates prior to panning. Liver infiltrating cell populations could be studied by FACS staining prior to panning. FIG. 14(A) is a table showing the absolute number of liver infiltrating cells per liver prior to panning, as well as the percentages of different populations in-vivo. FIG. 14(B) is a table showing the calculated absolute numbers of different cell types per liver of mice in each treatment group. FIG. 14(C) shows FACS analysis of CD4 vs. CD8 of liver infiltrating cells prior to anti-CD8 panning. FIG. 14(D) shows FACS analysis of CD3 vs. DX5 of liver infiltrating cells prior to anti-CD8 panning. FIG. 14(E) shows FACS analysis of B220 vs. CD11c of liver infiltrating cells prior to anti-CD8 panning.

In order to still further investigate the mechanism of action of Listeria augmentation in the hepatic tumor vaccine system, we analyzed the expression of interferon-gamma (IFN-γ) and Interleukin-10 (IL-10), which are cytokines that regulate immune-mediated inflammation. FIG. 8 shows the results of RT-PCR analysis of liver infiltrating, AH1-specific CD8 T-cells for IFN-γ and IL-10 expression. The results show that Listeria augmentation appears to be associated with increased production of IFN-γ and decreased production of IL-10 (notably, IL-10 is associated with inhibition of mononuclear phagocytes). In this experiment mice were given hepatic tumor challenge on day 0. Mice were treated with either vaccination alone initiated on day+3 or combination vaccination and Listeria on day+6. All mice received a booster vaccine on day+6. Mice were sacrificed on day 14, and liver infiltrating T-cells were isolated by straining through a 100 micron screen and filtering on a Percoll density gradient. The isolated cells were panned once using anti-CD8 antibody coated pans, and the adherent CD8 T-cells isolated from the 10 mice within each treatment group were pooled. The T-cells were stained using CD4-FITC, B220-FITC, CD8-cy, and AH1-loaded, PE conjugated Ld-Ig tetramer. The AH1 specific, CD8 T-cells were then isolated using a cell sorter, and analyzed via RT-PCR for expression of IFN-γ and IL-10.

Equivalents and Incorporation by Reference

Those skilled in the art will recognize, or be able to ascertain using no more than routine experimentation, numerous equivalents to the specific polypeptides, nucleic acids, cells, formulation, methods, assays and reagents described herein. Such equivalents are considered to be within the scope of this invention and are covered by the following claims.

The instant application includes numerous citations to learned texts, published articles and patent applications as well as issued U.S. and foreign patents. The entire contents of all of these citations are hereby incorporated by reference herein.

We claim:

1) A method of generating a systemic immune response against an organ or tissue-specific disease or condition in a subject comprising:
administering a therapeutically effective amount of a vaccine which generates an immune response against the organ or tissue-specific disease or condition; and
administering an agent that tropically localizes or is administered directly to the organ or tissue and that generates a localized immune response at the organ or tissue, thereby generating a systemic immune response against he organ or tissue-specific disease or condition.

2) The method of claim 1, wherein the organ or tissue-specific disease or condition is a tumor or cancerous growth and wherein the vaccine is a tumor vaccine.

3) The method of claim 2, wherein the tumor or cancerous growth is a liver cancer.

4) The method of claim 3, wherein the vaccine is an attenuated tumor cell line expressing GM-CSF.

5) The method of any of claims 1-4, in which the agent that tropically localizes to the organ or tissue is selected from the group consisting of: a virus, a bacterium, a yeast or a fungus with a natural tropism for the specific organ or tissue.

6) The method of any of claims 1-4, in which the agent that tropically localizes is an attenuated strain of Listeria monocytogenes.

7) The method of claim 5, wherein the organism tropically localizes to neovascular endothelium.

8) The method of any of claims 1-4, in which the agent is genetically engineered to tropically localize to the organ or tissue and is an organism selected from the group consisting of: a virus, a bacterium, a yeast or a fungus.

9) The method of claim 8, wherein the genetically engineered organism expresses a ligand for a receptor expressed by the organ or tissue.

10) The method of claim 9, wherein the ligand for the organ or tissue receptor has been fused to an envelope or coat protein of the organism.

11) The method of claim 8, wherein the organ or tissue is neovascular endothelium.

12) The method of claim 9, wherein the genetically engineered organism expresses a ligand for a receptor expressed by neovascular endothelium.

13) The method of claim 1, wherein the agent is an organism without natural tropism that is administered directly to the organ or tissue by a physical means selected from the group consisting of: direct injection, percutaneous catheter, surgery, and closed loop perfusion.

14) The method of claim 1, wherein the organ or tissue is the lungs and the method of administration is inhalation.

15) The method of claim 1, wherein the organ or tissue is the lungs and the method of administration is ingestion.

16) The method of claim 1, wherein the agent that tropically localizes or is administered directly to the organ or tissue is a genetically engineered organism that produces an activator of immunity or inflammation selected from the group consisting of: a chemokine, a cytokine, and an adhesion molecule.

17) The method of claim 1, wherein the agent that tropically localizes or is administered directly to the organ or tissue is an inflammatory agent.

18) A method of treating a tumor or cancerous growth localized to a tissue or organ in a subject comprising:
administering a therapeutically effective amount of a tumor vaccine which generates an immune response against the tumor or cancerous growth; and
administering an agent that tropically localizes or is administered directly to the organ or tissue and that generates a localized immune response at the organ or tissue, thereby treating the tumor or cancerous growth localized to the tissue or organ in the subject.

19) The method of claim 18, wherein the tumor or cancerous growth is a hepatic tumor, the tumor vaccine is a GM-CSF secreting whole tumor cell vaccine, and the agent that tropically localizes to the affected organ or tissue is an attenuated strain of Listeria monocytogenes.

20) The method of claim 18, wherein the tumor vaccine is a DNA tumor vaccine.

21) A formulation for generating a systemic immune response against an organ or tissue-specific disease or condition in a subject comprising a therapeutically effective amount of a vaccine which generates an immune response against the organ or tissue-specific disease or condition; and an agent that tropically localizes or is administered directly to the organ or tissue and that generates a localized immune response at the organ or tissue.

22) A formulation for treating a tumor or cancerous growth localized to a tissue or organ in a subject comprising a therapeutically effective amount of a tumor vaccine which generates an immune response against the tumor or cancerous growth; and an agent that tropically localizes or is administered directly to the organ or tissue and that generates a localized immune response at the organ or tissue.

23) The formulation of claim 21 or 22, wherein the agent that tropically localizes or is administered directly to the organ or tissue and that generates a localized immune response at the organ or tissue is an attenuated bacteria.

24) The formulation of claim 23, wherein the attenuated bacteria is an HIV-gag attenuated Listeria monocytogenes.

25) A kit for generating a systemic immune response against an organ or tissue-specific disease or condition in a subject comprising:
a vaccine which generates an immune response against the organ or tissue-specific disease or condition; and
an agent that tropically localizes or is administered directly to the organ or tissue and that generates a localized immune response at the organ or tissue.

26) A kit for treating a tumor or cancerous growth localized to a tissue or organ in a subject comprising:
a tumor vaccine which generates an immune response against the tumor or cancerous growth; and
an agent that tropically localizes or is administered directly to the organ or tissue and that generates a localized immune response at the organ or tissue.
